(19) United States
(12) Patent Application Publication
(10) Pub. No.: US 2025/0045834 A1
(43) Pub. Date: Feb. 6, 2025

(54) ENVIRONMENTAL ATTRIBUTES FOR AQUEOUS POLYMERIC COMPOSITION

(71) Applicant: BASF SE, Ludwigshafen Am Rhein (DE)

(72) Inventors: Bastian GRUMBRECHT, Ludwigshafen (DE); Steffen Thomas KLOSTERHALFEN, Ludwigshafen (DE); Christian KRUEGER, Ludwigshafen (DE); Ana ALBA PEREZ, Ludwigshafen am Rhein (DE); Martin BINDER, Ludwigshafen (DE); Alessandro PISTILLO, Ludwigshafen (DE); Martin KLATT, Ludwigshafen (DE); Christopher Alec ANDERLOHR, Ludwigshafen (DE); Kurt WENZL, Ludwigshafen (DE)

(21) Appl. No.: 18/717,698

(22) PCT Filed: Dec. 19, 2022
(86) PCT No.: PCT/EP22/86775

§ 371 Date: Jun. 7, 2024

Related U.S. Application Data
(60) Provisional application No. 63/416,091, filed on Oct. 14, 2022.

(30) Foreign Application Priority Data
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216326.5
Dec. 21, 2021 (EP) 21216327.3
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G06Q 40/06; C08F 218/08; C08F 236/10; G16C 20/90

(52) U.S. Cl. CPC G06Q 40/06; C08F 218/08; C08F 236/10; G16C 20/90

(57) ABSTRACT

Described herein are systems for producing an aqueous polymeric composition associated with a digital asset, methods for producing an aqueous polymeric composition associated with a digital asset, apparatuses for generating a digital asset, computer-implemented methods for generating a chemical passport, computer program elements for generating a digital asset, uses of an aqueous polymeric composition associated with a digital asset, uses of a digital asset, products produced from the aqueous polymeric composition and associated with a digital asset, a digital asset including one or more decentral identifier(s) and data related to the environmental impact data, apparatuses for producing a product associated with the digital asset and methods for producing a product associated with the digital asset.

### TECHNICAL FIELD

The present disclosure relates to systems for producing an aqueous polymeric composition associated with an aqueous polymeric composition passport or digital asset, methods for producing an aqueous polymeric composition associated with an aqueous polymeric composition passport or digital asset, apparatuses for generating an aqueous polymeric composition passport or digital asset, computer-implemented methods for generating a chemical passport, computer program elements for generating an aqueous polymeric composition passport or digital asset, uses of an aqueous polymeric composition associated with an aqueous polymeric composition passport or digital asset, uses of an aqueous polymeric composition passport or digital asset, products produced from the aqueous polymeric composition and associated with an aqueous polymeric composition passport or digital asset, an aqueous polymeric composition, an aqueous polymeric composition passport or digital asset including one or more decentral identifier(s) and data related to the environmental impact data, apparatuses for producing a product associated with the aqueous polymeric composition passport or digital asset and methods for producing a product associated with the aqueous polymeric composition passport or digital asset.

### TECHNICAL BACKGROUND

In supply chains the environmental impact of each supply chain participants is of great interest. Specifically in the field of chemistry, aqueous polymeric compositions are employed for a wide range of applications and are supplied to diverse value chains. In such complex systems transparency between value chain participants is hard to achieve.

### SUMMARY OF THE INVENTION

In one aspect disclosed is a system for producing an aqueous polymeric composition associated with an aqueous polymeric composition passport or a digital asset, the system comprising:
a chemical production network configured to produce the aqueous polymeric composition wherein the aqueous polymeric composition is produced from one or more input material(s) through one or more chemical process(s) of the chemical production network, wherein the one or more input material(s) and/or the one or more chemical process(s) are associated with environmental attribute(s);
a production operating apparatus configured to generate the aqueous polymeric composition passport or digital asset by
   providing a decentral identifier associated with the produced aqueous polymeric composition and one or more environmental attribute(s) of the one or more input material(s) and/or the one or more chemical process(s);
   linking the decentral identifier and the environmental attribute(s);
a chemical production network or system configured to provide each produced aqueous polymeric composition in association with the digital asset.

In another aspect disclosed is a system for producing an aqueous polymeric composition associated with an aqueous polymeric composition passport or digital asset, the system comprising:
a chemical production network configured to produce the aqueous polymeric composition from one or more input material(s) through chemical process(s), wherein the one or more input material(s) and/or the chemical process(s) are associated with environmental attribute(s);
a production operating apparatus configured to generate the aqueous polymeric composition passport or digital asset by providing and/or linking a decentral identifier associated with the aqueous polymeric composition and one or more environmental attribute(s) associated with the one or more input material(s) and/or the chemical process(s).

In another aspect disclosed is a method for producing an aqueous polymeric composition associated with an aqueous polymeric composition passport or digital asset, wherein the method comprises:
producing the aqueous polymeric composition from one or more input material(s) through one or more chemical process(s) of a chemical production network, wherein the one or more input material(s) and/or the one or more chemical process(s) are associated with environmental attribute(s);
generating the aqueous polymeric composition passport or digital asset by providing a decentral identifier associated with the produced aqueous polymeric composition and one or more environmental attribute(s) associated with the one or more input material(s) and/or the one or more chemical process(s);
   linking the decentral identifier and the one or more environmental attribute(s);
providing the produced aqueous polymeric composition in association with the aqueous polymeric composition passport or digital asset.

In another aspect disclosed is a method for producing an aqueous polymeric composition associated with an aqueous polymeric composition passport or a digital asset, wherein the method comprises:
producing the aqueous polymeric composition from one or more input material(s) through one or more chemical process(s) of a chemical production network, wherein the one or more input material(s) and/or the one or more chemical process(s) are associated with environmental attribute(s),
generating the aqueous polymeric composition passport or digital asset by providing and/or linking a decentral identifier associated with the aqueous polymeric composition and one or more environmental attribute(s) of the one or more input material(s) and/or the chemical process(s).

In another aspect disclosed is an apparatus for generating a passport or digital asset associated with an aqueous polymeric composition, wherein the aqueous polymeric composition is produced from one or more input material(s) through one or more chemical process(s) of a chemical production network, wherein the one or more input material(s) and/or the one or more chemical process(s) are associated with environmental attribute(s), the apparatus comprising:
providing a decentral identifier associated with the produced aqueous polymeric composition and one or more environmental attribute(s) of the one or more input material(s) and/or the one or more chemical process(s) used to produce the aqueous polymeric composition;
linking the decentral identifier and the environmental attribute(s);
providing the aqueous polymeric composition passport or digital asset in association with the produced aqueous polymeric composition e.g. to a decentral network, wherein the environmental attribute(s) associated with the aqueous polymeric composition is made accessible to a user of the aqueous polymeric composition through the aqueous polymeric composition passport or digital asset.

In another aspect disclosed is a method, e.g. a computer-implemented method, for generating an aqueous polymeric composition passport or digital asset associated with an aqueous polymeric composition, wherein the aqueous polymeric composition is produced from one or more input material(s) through one or more chemical process(s) of a chemical production network, wherein the one or more input material(s) and/or the one or more chemical process(s) are associated with environmental attribute(s), the method comprising:
providing a decentral identifier associated with the produced aqueous polymeric composition and one or more environmental attribute(s) of the one or more input material(s) and/or the one or more chemical process(s) used to produce the aqueous polymeric composition;
linking the decentral identifier and the environmental attribute(s);
providing the aqueous polymeric composition passport or digital asset in association with the produced aqueous polymeric composition e.g. to a decentral network, wherein the environmental attribute(s) associated with the aqueous polymeric composition is made accessible to a user of the aqueous polymeric composition through the aqueous polymeric composition passport or digital asset.

In another aspect disclosed is a computer element, such as a computer readable storage medium, a computer program or a computer program product, comprising instructions, which when executed by a computing node or a computing system, direct the computing node or computing system to carry out the steps of the computer-implemented methods disclosed herein.

In another aspect disclosed is a computer element, such as a computer readable storage medium, a computer program or a computer program product, comprising instructions, which when executed by the apparatuses disclosed herein, direct the apparatuses to carry out steps the apparatuses disclosed herein are configured to execute.

In another aspect disclosed is an aqueous polymeric composition associated with an aqueous polymeric composition passport or a digital asset as produced according to the methods disclosed herein. In another aspect disclosed is an aqueous polymeric composition associated with an aqueous polymeric composition passport or digital asset as produced according to the systems disclosed herein.

In another aspect disclosed is an aqueous polymeric composition associated with an aqueous polymeric composition passport or digital asset, wherein the aqueous polymeric composition is produced from one or more input material(s) through one or more chemical process(s) of a chemical production network, wherein the one or more input material(s) and/or the one or more chemical process(s) are associated with environmental attribute(s), wherein the aqueous polymeric composition passport or digital asset includes a decentral identifier associated with the produced aqueous polymeric composition and/or formulations using said aqueous polymeric compositions and a link to environmental attribute(s) associated with one or more environmental attribute(s) of one or more input material(s) and/or one or more chemical process(s) used to produce the aqueous polymeric composition.

In another aspect disclosed is an aqueous polymeric composition passport or digital asset as generated according to the methods disclosed herein. In another aspect disclosed is an aqueous polymeric composition passport or digital asset as generated according to the apparatuses disclosed herein.

In another aspect disclosed is a production system for producing a product from the aqueous polymeric composition associated with the aqueous polymeric composition passport or digital asset as provided according to the systems, apparatuses or methods disclosed herein. In another aspect disclosed is a production method for producing a product from the aqueous polymeric composition associated with the aqueous polymeric composition passport or digital asset as provided according to the systems, apparatuses or methods disclosed herein.

In another aspect disclosed is a use of the aqueous polymeric composition associated with the aqueous polymeric composition passport or digital asset as disclosed herein for producing a product from the aqueous polymeric composition associated with the aqueous polymeric composition passport or digital asset.

In another aspect disclosed is a use of the aqueous polymeric composition passport or digital asset as disclosed herein for generating a product passport or digital asset associated with a product produced from the aqueous polymeric composition associated with the aqueous polymeric composition passport or digital asset. In another aspect disclosed is a method for using the digital asset generated according to the methods disclosed herein in production of a product produced from the aqueous polymeric composition associated with the aqueous polymeric composition passport or digital asset.

In another aspect disclosed is an aqueous polymeric composition associated with a digital asset including a decentral identifier associated with the aqueous polymeric composition and linked to one or more environmental attribute(s) of the one or more input material(s) and/or the one or more chemical process(s) used to produce the aqueous polymeric composition.

In another aspect disclosed is a use of the aqueous polymeric composition associated with the aqueous polymeric composition passport or digital asset for producing a product from the aqueous polymeric composition and associating the aqueous polymeric composition passport or digital asset with the product produced from the aqueous polymeric composition. In another aspect disclosed is a use of the aqueous polymeric composition associated with the aqueous polymeric composition passport or digital asset for producing a product from the aqueous polymeric composition and deriving a product passport or digital asset from the aqueous polymeric composition passport or digital asset. In another aspect disclosed is a method for using the aqueous polymeric composition associated with the digital asset for producing a product from the aqueous polymeric composition as disclosed herein and deriving a digital asset associated with the product from the aqueous polymeric composition passport or Aqueous polymeric composition digital asset.

Any disclosure and embodiments described herein relate to the methods, the systems, chemical products, aqueous polymeric composition, aqueous polymeric composition passports, digital assets and the computer elements lined out above and below. Advantageously, the benefits provided by any of the embodiments and examples equally apply to all other embodiments and examples.

### EMBODIMENTS

In the following, embodiments of the present disclosure will be outlined by ways of embodiments and/or example. It is to be understood that the present disclosure is not limited to said embodiments and/or examples.

Determining, generating includes initiating or causing to determine, generate. Providing includes “initiating or causing to access, determine, generate, send or receive”. “Initiating or causing to perform an action” includes any processing signal that triggers a computing node to perform the respective action.

The methods, the systems, aqueous polymeric composition, aqueous polymeric composition passports, digital assets and the computer elements disclosed herein provide an efficient, secure and robust way for sharing or exchanging environmental impact data across different participant nodes in value chains. In particular, by providing Aqueous polymeric composition specific data via the aqueous polymeric composition passport or digital asset, environmental impacts can be shared and made transparent from the material to the product produced from such material. The Aqueous polymeric composition passport or digital asset enables secure data exchange, since data access can be controlled by the aqueous polymeric composition provider. The exchanged data assets can be specific to the aqueous polymeric composition as produced and tailored to the needs of the aqueous polymeric composition user. This way an improved tracking and tracing of Aqueous polymeric composition can be achieved by securely providing environmental impact data in diverse and highly complex value chains. The environmental impact of Aqueous polymeric composition can hence be tracked leading to simpler, more efficient and sustainable handling of Aqueous polymeric composition by value chain participants.

In an embodiment, aqueous polymeric compositions being associated with the composition passport or digital asset may be selected from compositions comprising at least one polymer made directly by polymerization in water phase or is produced in non-aqueous liquid phase with transfer into aqueous phase, typically but not exclusively, without prior separation.

Aqueous polymeric composition associated with the composition passport or digital asset may be selected from e.g. aqueous dispersions comprising emulsion polymers which comprise, in copolymerized form,
1. from 0.1 to 5% by wt. of at least one α,β-monoethylenically unsaturated
   mono- and/or dicarboxylic acid having from 3 to 6 carbon atoms and/or amide thereof and
from 50 to 99.9% by wt. of at least one ester of acrylic and/or methacrylic acid with alkanols having from 1 to 20 carbon atoms and/or styrene,
and optionally at least one further monomer
   or
2. from 0.1 to 5% by wt. of at least one α,β-monoethylenically unsaturated
   mono- and/or dicarboxylic acid having from 3 to 6 carbon atoms and/or amide thereof and
from 50 to 99.9% by wt. of styrene and/or butadiene,
and optionally at least one further monomer
   or
3. from 0.1 to 5% by wt. of at least one α,β-monoethylenically unsaturated
   mono- and/or dicarboxylic acid having from 3 to 6 carbon atoms and/or amide thereof and
from 50 to 99.9% by wt. of vinyl chloride and/or vinylidene chloride,
and optionally at least one further monomer
   or
4. from 0.1 to 5% by wt. of at least one α,β-monoethylenically unsaturated
   mono- and/or dicarboxylic acid having from 3 to 6 carbon atoms and/or amide thereof and
from 50 to 99.9% by wt. of vinyl acetate, vinyl propionate, vinyl esters of versatic acid, vinyl esters of long-chain fatty acids, and/or ethylene,
and optionally at least one further monomer.

Examples of α,β-monoethylenically unsaturated mono- and/or dicarboxylic acids include, but are not limited to monoethylenically unsaturated monocarboxylic acids having 3 to 6 carbon atoms, such as acrylic acid, methacrylic acid, crotonic acid, 2-ethylpropenoic acid, 2-propylpropenoic acid, 2-acryloxyacetic acid and 2-methacryloxyacetic acid;
monoethylenically unsaturated dicarboxylic acids having 4 to 6 carbon atoms, such as itaconic acid and fumaric acid
monoethylenically unsaturated sulfonic acids, such as vinylsulfonic acid, allylsulfonic acid, styrenesulfonic acid, 2-acrylamido-2-methylpropane sulfonic acid,
monoethylenically unsaturated phosphonic acids such as vinylphosphonic acid, allylphosphonic acid, styrenephosphonic acid and 2-acrylamido-2-methylpropane phosphonic acid,
monoethylenically unsaturated phosphoric acids such as monophosphates of hydroxyalkyl acrylates, monophosphates of hydroxyalkyl methacrylates, monophosphates of alkoxylated hydroxyalkyl acrylates and monophosphates of alkoxylated hydroxyalkyl methacrylates.

Esters of acrylic acid and/or methacrylic acid with alkanols having 1 to 20 carbon denominates a group of linear or branched saturated hydrocarbon radicals having from 1 to 20 carbon atoms. Examples of alkyl include but are not limited to methyl, ethyl, n-propyl, isopropyl, n-butyl, sec-butyl, isobutyl, tert-butyl, 2-methylpropyl (isopropyl), 1,1-dimethylethyl (tert-butyl), pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-heptyl, n-octyl, 2-octyl, 2-ethylhexyl, nonyl, isononyl, decyl, undecyl, dodecyl, tridecyl, isotridecyl, tetradecyl, pentadecyl, hexadecyl, heptadecyl, octadecyl, nonadecyl, eicosyl, heneicosyl docosyl and in case of nonyl, isononyl, decyl, undecyl, dodecyl, tridecyl, isotridecyl, tetradecyl, pentadecyl, hexadecyl, heptadecyl, octadecyl, nonadecyl, eicosyl, heneicosyl docosyl their isomers, in particular mixtures of isomers such as “isononyl”, “isodecyl”. Examples of C₁-C₄-alkyl are for example methyl, ethyl, propyl, 1-methylethyl, butyl, 1-methylpropyl, 2-methylpropyl or 1,1-dimethylethyl.

Further Monomers include, but are not limited to
C₅-C₂₀-cycloalkyl esters of (meth)acrylic acid including but are not limited to cyclohexylacrylate, cyclohexylmethacrylate, norbornylacrylate, norbornylmethacrylate, isobornylacrylate, isobornylmethacrylate, 1,3-dioxan-5-yl-acrylate, 1,3-dioxan-5-yl-methacrylate, 2,2-dimethyl-1,3-dioxan-5-yl-acrylate, 2,2-dimethyl-1,3-dioxan-5-yl-methacrylate; C₅-C₂₀-cycloalkylmethyl esters of (meth)acrylic acid including but are not limited to cyclohexylmethylacrylate, cyclohexylmethylmethacrylate, 1,3-dioxolan-4-yl-methyl acrylate, 1,3-dioxolan-4-ylmethyl methacrylate, 2,2-dimethyl-1,3-dioxolan-4-ylmethyl acrylate, 2,2-dimethyl-1,3-dioxolan-4-ylmethyl methacrylate, oxolan-2-yl-methyl acrylate (tetrahydrofurfuryl acrylate) and oxolan-2-yl-methyl methacrylate (tetrahydrofurfuryl methacrylate);
further monovinyl aromatic monomers, such as 2-methylstyrene, 4-methylstyrene;
monomers which have a functional group selected from hydroxyalkyl groups, in particular hydroxy-C₂-C₄-alkyl group, a primary carboxamide group, urea groups and keto groups, for example acrylamide and methacrylamide, C₁-C₄-alkyl esters of acrylic acid or methacrylic acid and the N—C₁-C₄-alkyl amides of acrylic acid or methacrylic acid, where the C₁-C₄-alkyl group bears an urea group or a 2-oxoimidazolin group such as 2-(2-oxo-imidazolidin-1-yl)ethyl acrylate, 2-(2-oxoimidazolidin-1-yl)ethyl methacrylate, which are also termed 2-ureido acrylate and 2-ureido methacrylate, respectively, N-(2-acryloxyethyl)urea, N-(2-methacryloxyethyl)urea, N-(2-(2-oxoimidazolidin-1-yl)ethyl) acrylamide, N-(2-(2-oxo-imidazolidin-1-yl)ethyl) methacrylamide, as well as allyl or vinyl substituted ureas and allyl or vinyl substituted 2-oxoimidazolin compounds such as 1-allyl-2-oxoimidazolin, N-allyl urea and N-vinylurea; C₂-C₈-oxoalkyl esters of acrylic acid or methacrylic acid and the N—C₂-C₈-oxoalkyl amides of acrylic acid or methacrylic acid, such as diacetoneacrylamide (DAAM), and diacetonemethacrylamide, C₁-C₄-alkyl esters of acrylic acid or methacrylic acid and the N—C₁-C₄-alkyl amides of acrylic acid or methacrylic acid, where the C₁-C₄-alkyl group bears a 2-acetylacetoxy group of the formula O—C(═O)—CH₂—C(═O)—CH₃ (also termed acetoacetoxy group), such as acetoacetoxyethyl acrylate, acetoacetoxypropyl methacrylate, acetoacetoxybutyl methacrylate and 2-(acetoacetoxy)ethyl methacrylate.
monoethylenically unsaturated silan functional monomers, e.g. monomers which in addition to an ethylenically unsaturated double bond bear at least one mono-, di- and/or tri-C₁-C₄-alkoxysilane group, such as vinyl trimethoxysilane, vinyl triethoxysilane, methacryloxyethyl trimethoxysilane, methacryloxyethyl triethoxysilane, and mixtures thereof
unsaturated monomers bearing at least one epoxy group (monomers M6b), in particular a glycidyl group such as glycidyl acrylate, glycidyl methacrylate, 2-glycidyloxyethyl acrylate and 2-glycidyloxyethyl methacrylate
multiethylenically unsaturated monomers e.g. diesters of monoethylenically unsaturated C₃-C₆ monocarboxylic acids with saturated aliphatic or cycloaliphatic diols, in particular diesters of acrylic acid or methacrylic acid, such as the diacrylates and the dimethacrylates of ethylene glycol (1,2-ethanediol), propylene glycol (1,2-propanediol), 1,2-butanediol, 1,3-butanediol, 1,4-butanediol, neopentyl glycol (2,2-dimethyl-1,3-propanediol), 1,6-hexanediol and 1,2-cyclohexanediol; monoesters of monoethylenically unsaturated C₃-C₆ monocarboxylic acids with monoethylenically unsaturated aliphatic or cycloaliphatic monohydroxy compounds, such as the acrylates and the methacrylates of vinyl alcohol (ethenol), allyl alcohol (2-propen-1-ol), 2-cyclohexen-1-ol or norbornenol, such as allyl acrylate and allyl methacrylate; and divinyl aromatic compounds, such as 1,3-divinyl benzene, 1,4-divinyl benzene.

Further the polymeric composition used can be an aqueous dispersions comprising a polyurethane synthesized from
a) diisocyanates,
b) diols, of which
b1) from 10 to 100 mol %, based on the total amount of diols (b), have a molecular weight of from 500 to 5000, and
b2) from 0 to 90 mol %, based on the total amount of diols (b), have a molecular weight of from 60 to 500 g/mol,
c) monomers other than the monomers (a) and (b) having at least one isocyanate group or at least one group which is reactive toward isocyanate groups and which, moreover, carry at least one hydrophilic group or a potentially hydrophilic group, which gives rise to the dispersibility of the polyurethanes in water,
d) optionally, further polyvalent compounds which differ from the monomers (a) to (c) which have reactive groups, which groups are alcoholic hydroxyl groups, primary or secondary amino groups or isocyanate groups, and
e) optional monofunctional compounds distinct from monomers (a) to (d) and comprising a reactive group which is an alcoholic hydroxyl group, a primary or secondary amino group or an isocyanate group, obtainable by reacting the monomers a), b), c) and optionally d) and e) in the presence of a catalyst, e.g. a tin salt, such as dibutyltin dilaurate (DE-A 199 59 6539) or tin-free catalysts, for example based on bismuth neodecanoate (e.g. Borchikat® 315 from OMG Borchers GmbH, Langenfeld, Germany).

The aqueous dispersions according to the invention comprise polyurethanes which in addition to other monomers are derived from diisocyanates a), preference being given to using such diisocyanates a) as are typically employed in polyurethane chemistry. Suitable monomers (a) include in particular diisocyanates of formula X (NCO)2 where X is an aliphatic hydrocarbon radical comprising from 4 to 12 carbon atoms, a cycloaliphatic or aromatic hydrocarbon radical comprising from 6 to 15 carbon atoms or an araliphatic hydrocarbon radical comprising from 7 to 15 carbon atoms. Examples of such diisocyanates include tetramethylene diisocyanate, hexamethylene diisocyanate (HDI), dodecamethylene diisocyanate, 1,4-diisocyanatocyclohexane, 1-isocyanato-3,5,5-trimethyl-5-isocyanatomethylcyclohexane (IPDI), 2,2-bis(4-isocyanatocyclohexyl)propane, trimethylhexane diisocyanate, 1,4-diisocyanatobenzene, 2,4-diisocyanatotoluene, 2,6-diisocyanatotoluene, 4,4′-diisocyanatodiphenylmethane, 2,4-diisocyanatodiphenylmethane, p-xylylene diisocyanate, tetramethylxylylene diisocyanate (TMXDI), the isomers of bis(4-isocyanatocyclohexyl)methane (HMDI) such as the trans/trans, cis/cis and cis/trans isomers and mixtures consisting of these compounds.

Such diisocyanates are commercially available. Mixtures of these isocyanates of particular importance are mixtures of the respective structural isomers of diisocyanatotoluene and diisocyanatodiphenylmethane, the mixture of 80 mol % of 2,4-diisocyanatotoluene and 20 mol % of 2,6-diisocyanatotoluene being particularly suitable.

Furthermore, the mixtures of aromatic isocyanates, such as 2,4-diisocyanatotoluene and/or 2,6-diisocyanatotoluene, with aliphatic or cycloaliphatic isocyanates,

such as hexamethylene diisocyanate or IPDI, are particularly advantageous, the preferred mixing ratio of the aliphatic isocyanate to the aromatic isocyanate being from 4:1 to 1:4. In addition to the abovementioned compounds, the synthesis of the polyurethanes may also employ isocyanates bearing further capped isocyanate groups, for example uretdione groups, in addition to the free isocyanate groups. With a view to achieving good film formation and elasticity, suitable diols (b) especially include relatively high molecular weight diols (b1) having a molecular weight of about 500 to 5000 g/mol, preferably about 1000 to 3000 g/mol. The diols (b1) are, in particular, polyester polyols, which are known from, for example, Ullmanns Encyklopädie der technischen Chemie (Ullmann's Encyclopedia of Industrial Chemistry), 4th edition, Volume 19, pp. 62 to 65. Preference is given to using polyester polyols obtained by reaction of dihydric alcohols with dibasic carboxylic acids. In the place of the free polycarboxylic acids, it is also possible to produce the polyester polyols using the corresponding polycarboxylic anhydrides or the corresponding polycarboxylic acid esters of lower alcohols or their mixtures.

As sulfonated polyester polyols it is also possible to employ, for example, the compounds disclosed in EP 2 666 800, for example the product “SS55-225-130”, a sulfonated polyesterdiol comprising free sodium sulfonate groups, molecular weight 550; Crompton Corp., Middlebury CT. The polycarboxylic acids may be aliphatic, cycloaliphatic, araliphatic, aromatic or heterocyclic and may optionally be substituted, for example by halogen atoms, and/or be unsaturated. Examples thereof which may be mentioned are: suberic acid, azelaic acid, phthalic acid, isophthalic acid, phthalic anhydride, tetrahydrophthalic anhydride, hexahydrophthalic anhydride, tetrachlorophthalic anhydride, endomethylenetetrahydrophthalic anhydride, glutaric anhydride, maleic acid, maleic anhydride, fumaric acid, dimeric fatty acids and dimethylsulfoisophthalic acid. Preference is given to dicarboxylic acids of the general formula HOOC(CH2)y-COOH where y is a number from 1 to 20, preferably an even number from 2 to 20, for example succinic acid, adipic acid, sebacic acid and dodecanedicarboxylic acid. Suitable polyhydric alcohols include, for example, ethylene glycol, propane-1,2-diol, propane-1,3-diol, butane-1,3-diol, butene-1,4-diol, butyne-1,4-diol, pentane-1,5-diol, neopentyl glycol, bis(hydroxymethyl)cyclohexanes such as 1,4-bis(hydroxymethyl)cyclohexane, 2-methylpropane-1,3-diol, methylpentanediols and also diethylene glycol, triethylene glycol, tetraethylene glycol, polyethylene glycol, dipropylene glycol, polypropylene glycol, dibutylene glycol and polybutylene glycols. Preference is given to alcohols of general formula HO—(CH2)x-OH where x is a number from 1 to 20, preferably an even number from 2 to 20. Examples thereof include ethylene glycol, butane-1,4-diol, hexane-1,6-diol, octane-1,8-diol and dodecane-1,12-diol. Neopentyl glycol is also preferred.

Also suitable are polycarbonate diols, obtainable, for example, by reaction of phosgene with an excess of the low molecular weight alcohols cited as synthesis components for the polyester polyols. Also suitable are lactone-based polyester diols which are homo- or copolymers of lactones, preferably addition products of lactones having terminal hydroxyl groups onto suitable difunctional starter molecules. Suitable lactones are preferably lactones derived from compounds of general formula HO—(CH2)z-COOH where z is a number from 1 to 20 and one H atom of a methylene unit may also be substituted by a C1 to C4 alkyl radical. Examples include ε-caprolactone, β-propiolactone, γ-butyrolactone and/or methyl-ε-caprolactone and mixtures thereof. Suitable starter components are, for example, the low molecular weight dihydric alcohols cited hereinabove as a synthesis component for the polyester polyols. The corresponding polymers of ε-caprolactone are particularly preferred. Lower polyester diols or polyether diols may also be used as starters to prepare the lactone polymers. Instead of the polymers of lactones, it is also possible to use the corresponding chemically equivalent polycondensates of the hydroxycarboxylic acids corresponding to the lactones.

Suitable monomers (b1) further include polyether diols. Said polyether diols are in particular obtainable by polymerization of ethylene oxide, propylene oxide, butylene oxide, tetrahydrofuran, styrene oxide or epichlorohydrin with itself, for example in the presence of BF₃ or by addition of these compounds, optionally mixed or in succession, onto starting components having reactive hydrogen atoms, such as alcohols or amines, for example water, ethylene glycol, propane-1,2-diol, propane-1,3-diol, 1,2-bis(4-hydroxydiphenyl)propane or aniline. Particular preference is given to polytetrahydrofuran having a molecular weight of from 240 to 5000, especially from 500 to 4500. Mixtures of polyester diols and polyether diols may also be used as monomers (b1). Likewise suitable are polyhydroxyolefins, preferably those comprising 2 terminal hydroxyl groups, for example α,ω-dihydroxypolybutadiene, α,ω-dihydroxy polymethacrylic ester or α,ω-dihydroxy polyacrylic ester as monomers (c1). Such compounds are disclosed in EP-A 622 378 for example. Further suitable polyols are polyacetals, polysiloxanes and alkyd resins.

The polyols may also be employed as mixtures in a ratio of from 0.1:1 to 1:9.

The hardness and the modulus of elasticity of the polyurethanes can be increased when, besides the diols (b1), low-molecular-weight diols (b2) with a molecular weight of approximately 60 to 500, preferably of from 62 to 200 g/mol, are additionally employed as diols (b).

Monomers (b2) which are employed are, mainly, the structural components of the short-chain alkanediols which have been mentioned for the preparation of polyester polyols, with diols having 2 to 12 C atoms, unbranched diols having 2 to 12 C atoms and an even number of C atoms, and pentane-1,5-diol and neopentyl glycol being preferred.

Preferably, the amount of the diols (b1), based on the total amount of the diols (b), is from 10 to 100 mol % and the amount of the monomers (b2), based on the total amount of the diols (b), is from 0 to 90 mol %. The ratio of the diols (b1) to the monomers (b2) is particularly preferably 0.1:1 to 5:1, more preferably 0.2:1 to 2:1.

In order to ensure that the polyurethanes are water-dispersible, the polyurethanes are synthesized not only from components (a), (b) and optionally (d) but also from monomers (c) which are distinct from components (a), (b) and (d) and which bear at least one isocyanate group or at least one isocyanate-group reactive group and moreover bear at least one hydrophilic group or a group which can be converted into a hydrophilic group.

Hereinbelow, the term “hydrophilic groups or potentially hydrophillic groups” is abbreviated to “(potentially) hydrophilic groups”. The (potentially) hydrophilic groups react with isocyanates substantially more slowly than the functional groups of the monomers used to synthesize the polymer main chain.

The proportion of components comprising (potentially) hydrophilic groups in the total amount of components (a), (b), (c), (d) and (e) is generally measured such that the molar amount of the (potentially) hydrophilic groups based on the amount by weight of all monomers (a) to (e) is from 30 to 1000, preferably from 50 to 500 and more preferably from 80 to 300 mmol/kg.

The (potentially) hydrophilic groups may be nonionic or preferably (potentially) ionic hydrophilic groups. Suitable nonionic hydrophilic groups include in particular polyethylene glycol ethers composed of preferably from 5 to 150 and preferably from 40 to 120 ethylene oxide repeating units. The content of polyethylene oxide units is generally from 0.1 to 15 and preferably from 1 to 10 wt % based on the amount by weight of all monomers (a) to (e).

Preferred monomers comprising nonionic hydrophilic groups are polyethylene oxide diols, polyethylene oxide monools, and the reaction products of a polyethylene glycol and a diisocyanate which bear a terminally etherified polyethylene glycol radical. Such diisocyanates and processes for the preparation thereof are described in patent specifications U.S. Pat. Nos. 3,905,929 and 3,920,598.

Ionic hydrophilic groups are especially anionic groups such as sulfonate, carboxylate and phosphate groups in the form of the alkali metal or ammonium salts thereof and also cationic groups such as ammonium groups, in particular protonated tertiary amino or quaternary ammonium groups.

Potentially ionic hydrophilic groups are especially those which may be converted into the abovementioned ionic hydrophilic groups by simple neutralization, hydrolysis or quaternization reactions, i.e., carboxylic acid groups, or tertiary amino groups for example.

(Potentially) ionic monomers (c) are described, for example, in Ullmanns Encyklopädie der technischen Chemie (Ullmann's Encyclopedia of Industrial Chemistry), 4th edition, Volume 19, pp. 311-313 and are described in detail, for example, in DE-A 14 95 745.

(Potentially) cationic monomers (c) of particular practical importance are especially monomers comprising tertiary amino groups, for example: tris(hydroxyalkyl)amines, N,N′-bis(hydroxyalkyl)alkylamines, N-hydroxyalkyldialkylamines, tris(aminoalkyl)amines, N,N′-bis(aminoalkyl)alkylamines, N-aminoalkyldialkylamines, the alkyl radicals and alkanediyl units of these tertiary amines consisting independently of from 1 to 6 carbon atoms. Also suitable are polyethers comprising tertiary nitrogen atoms and preferably two terminal hydroxyl groups, as obtainable in a conventional manner, for example, by alkoxylation of amines comprising two hydrogen atoms attached to amine nitrogen, for example methylamine, aniline or N,N′-dimethylhydrazine. Such polyethers generally have a molar weight of between 500 and 6000 g/mol.

These tertiary amines are converted into the ammonium salts, either with acids, preferably strong mineral acids such as phosphoric acid, sulfuric acid, hydrohalic acids, or strong organic acids or by conversion with suitable quaternization agents such as C₁-to C6-alkyl halides or benzyl halides, for example bromides or chlorides.

Suitable monomers comprising (potentially) anionic groups typically include aliphatic, cycloaliphatic, araliphatic or aromatic carboxylic acids and sulfonic acids bearing at least one alcoholic hydroxyl group or at least one primary or secondary amino group. Preferred are dihydroxyalkylcarboxylic acids, especially those having 3 to 10 carbon atoms, as they are also described in U.S. Pat. No. 3,412,054. Particular preference is given to compounds of general formula (c1)

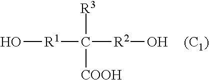

where R¹ and R² represent a C1 to C4 alkanediyl unit and R³ represents a C1 to C4 alkyl unit, dimethylolpropionic acid (DMPA) being especially preferred. Also suitable are corresponding dihydroxysulfonic acids and dihydroxyphosphonic acids such as 2,3-dihydroxypropanephosphonic acid.

Otherwise suitable are dihydroxyl compounds having a molecular weight from over 500 to 10 000 g/mol and comprising at least 2 carboxylate groups, which are disclosed in DE-A 39 11 827. They are obtainable by reacting dihydroxyl compounds with tetracarboxylic dianhydrides, such as pyromellitic dianhydride or cyclopentanetetracarboxylic dianhydride in the molar ratio 2:1 to 1.05:1 in a polyaddition reaction. Suitable dihydroxy compounds are in particular the monomers (b2) cited as chain extenders and diols (b1).

Suitable monomers (c) comprising isocyanate-reactive amino groups are aminocarboxylic acids such as lysine, β-alanine or the adducts, cited in DE-A 20 34 479, of aliphatic diprimary diamines onto α,β-unsaturated carboxylic or sulfonic acids.

Such compounds, for example, comply with the formula (c2)

H₂N—R⁴—NH—R⁵—X (c2)

where R⁴ and R⁵ are independently a C₁ to C₆ alkanediyl unit, preferably ethylene, and X is —COOH or —SO₃H.

Particularly preferred compounds of formula (c2) are N-(2-aminoethyl)-2-aminoethanecarboxylic acid and N-(2-aminoethyl)-2-aminoethanesulfonic acid and the corresponding alkali metal salts, sodium being particularly preferred as counterion. Also preferred are the adducts of the abovementioned aliphatic diprimary diamines onto 2-acrylamido-2-methylpropanesulfonic acid as are described in DE patent specification 19 54 090 for example. Further suitable aminosulfonic acids are, for example, sodium 2-((2-aminoethyl)amino)ethanesulfonate, diaminoalkylsulfonic acid and the salts thereof, for example ethylenediamino-β-ethylsulfonic acid, ethylenediaminopropylsulfonic or ethylenediaminobutylsulfonic acid, 1,2- or, 1,3-propylenediamino-β-ethylsulfonic acid. Provided that monomers comprising potentially ionic groups are employed, the conversion thereof into the ionic form may be effected before, during but preferably after the isocyanate polyaddition since the ionic monomers are often only sparingly soluble in the reaction mixture. The sulfonate or carboxylate groups are especially preferably present in the form of their salts with an alkali metal ion or with an ammonium ion as counterion.

The monomers (d) which are distinct from monomers (a) to (c) and which are optionally also constituents of the polyurethane generally serve to crosslink or to chain-extend. In general, they are more than dihydric/nonphenolic alcohols, amines having 2 or more primary and/or secondary amino groups and compounds which, besides one or more alcoholic hydroxyl groups, include one or more primary and/or secondary amino groups.

Alcohols having a hydricity greater than 2 and which may be used to establish a certain degree of branching or crosslinking are, for example, trimethylolpropane, glycerol and sugar.

Also suitable are monoalcohols which, in addition to the hydroxyl group, bear a further isocyanate-reactive group such as monoalcohols comprising one or more primary and/or secondary amino groups, e.g. monoethanolamine. Polyamines with 2 or more primary and/or secondary amino groups are primarily used when the chain extension and/or crosslinking is to take place in the presence of water since amines generally react with isocyanates more rapidly than do alcohols or water.

This is often necessary when aqueous dispersions of crosslinked polyurethanes or polyurethanes of high molecular weight are desired. The procedure in such cases comprises preparing prepolymers comprising isocyanate groups, rapidly dispersing said prepolymers in water and subsequently chain-extending or crosslinking said prepolymers by adding compounds comprising a plurality of isocyanate-reactive amino groups.

Amines which are suitable for this purpose are, in general, polyfunctional amines in the molecular weight range of from 32 to 500 g/mol, preferably from 60 to 300 g/mol, which comprise at least two amino groups selected from the group of the primary and secondary amino groups. Examples thereof include diamines such as diaminoethane, diaminopropanes, diaminobutanes, diaminohexanes, piperazine, 2,5-dimethylpiperazine, amino-3-aminomethyl-3,5,5-trimethylcyclohexane (isophoronediamine, IPDA), 4,4′-diaminodicyclohexylmethane, 1,4-diaminocyclohexane, aminoethylethanolamine, hydrazine, hydrazine hydrate or triamines such as diethylenetriamine or 1,8-diamino-4-aminomethyloctane. The amines may also be employed in blocked form, for example in the form of the corresponding ketimines (see, for example, CA-A 1 129 128), ketazines (cf., for example, U.S. Pat. No. 4,269,748) or amine salts (see U.S. Pat. No. 4,292,226). Oxazolidines are used, for example, in U.S. Pat. No. 4,192,937, too, as capped polyamines which can be employed for synthesizing the polyurethanes according to the invention for extending the chains of the prepolymers. Use of such capped polyamines generally comprises mixing said polyamines with the prepolymers in the absence of water and subsequently mixing this mixture with the dispersing water or a portion of the dispersing water, thus releasing the corresponding polyamines hydrolytically. It is preferred to use mixtures of di- and triamines; it is especially preferred to use mixtures of isophorone diamine (IPDA) and diethylene triamine (DETA).

The polyurethanes preferably comprise from 1 to 30 and more preferably from 4 to 25 mol %, based on the total amount of components (b) and (d), of a polyamine comprising at least 2 isocyanate-reactive amino groups as monomers (d). Alcohols having a hydricity greater than 2 and which may be used to establish a certain degree of branching or crosslinking are, for example, trimethylolpropane, glycerol and sugar. Monomers (d) higher than difunctional isocyanates may also be used for the same purpose. Commercially available compounds are, for example, the isocyanurate or the biuret of hexamethylene diisocyanate.

Monomers (e) which are optionally co-used are monoisocyanates, monoalcohols and monoprimary and -secondary amines. The proportion thereof is generally no more than 10 mol % based on the total molar amount of the monomers. These monofunctional compounds typically bear further functional groups such as olefinic groups or carbonyl groups and serve to introduce functional groups into the polyurethane which render possible the dispersal or crosslinking or further polymer-analogous reaction of the polyurethane. Suitable for this purpose are monomers such as isopropenyl-α,α-dimethylbenzyl isocyanate (TMI) and esters of acrylic or methacrylic acid such as hydroxyethyl acrylate, hydroxyethyl methacrylate, hydroxypropyl acrylate and hydroxypropyl methacrylate.

Further the polymeric composition used can be an aqueous dispersions synthesized from an aqueous polyurethane-polyacrylate hybrid dispersion

obtainable by radical polymerization of an acrylate polymer in the presence of at least one polyurethane and at least one initiator system, wherein the at least one polyurethane has a content of polyalkylene oxide of at least 10 g/kg of polyurethane and a content of a sulfonated raw material of at least 25 mmol per kg of polyurethane and the acrylate polymer has a glass transition temperature of from −50° C. to 50° C. and the mass fraction of the polyurethane is at least 5% and no more than 95% based on the overall hybrid polymer.

In a further aspect disclosed is a system for producing an formulation using said aqueous polymeric composition associated with an formulation passport or a digital asset.

In a further aspect disclosed is a system for producing a polymer powder obtainable by drying said aqueous polymeric composition associated with an formulation passport or a digital asset.

In an embodiment, polymer powder obtainable by drying said aqueous polymeric composition being associated with the composition passport or digital asset may be selected from compositions containing at least one material produced by spray drying of a dispersion with or without use of spray drying adjuvants.

Drying adjuvants for producing redispersion powders and/or polymeric dispersants in powder form are water-soluble polymers comprising
a) monoethylenically unsaturated monomers carrying acidic groups in an acidic, or partially or completely neutralized form from the series acrylic acid, methacrylic acid, 2-ethylacrylic acid, 2-propylacrylic acid, vinylacetic acid, crotonic and isocrotonic acid, maleic acid, fumaric acid, itaconic acid, citraconic acid, vinylsulfonic acid, methallylsulfonic acid, 2-acrylamido-2-methylpropylsulfonic acid, 4-vinylphenylsulfonic acid, vinylphosphonic acid and (semi)esters and/or amides of the said carboxylic acids and mixtures and/or anhydrides thereof and optionally
b) other monomers that can copolymerize with the monomers of group a) from the series (meth)acrylnitrile, (meth)allyl alcohol, vinyl ethers such as hydroxy- and alkoxypolyethyleneoxy or -polypropyleneoxyvinyl ethers and analogous allyl ethers containing up to 10 repeating units such as e.g. polyethylene glycol monoallyl ethers or mixed ethylene oxide (EO)-propylene oxide (PO)-mono-allyl ethers, N-vinylacetamide, N-vinyllactams, N-vinylpyrrolidone, N-vinylimidazole, silicon-functional comonomers such as (meth-)acryloxyalkyl-tri-(alkoxy)silanes, vinyltrialkoxysilanes and vinylmethyldialkoxysilanes containing C1 to C5 alkyl or alkoxy residues and derivatives and mixtures thereof.

Within the scope of the present invention water-soluble polymers have proven to be particularly suitable which contain at least 20% by weight, preferably at least 30% by weight of monomers a) and at most 80% by weight of monomers b). The water-soluble polymers additionally preferably contain at least 5% by weight, in particular at least 10% by weight of monomers b).

Within the scope of the use according to the invention it has also proven to be advantageous when the acidic groups of the monoethylenically unsaturated monomers a) are at least partially neutralized before, during or after polymerization. The at least partial neutralization can in particular be carried out with the aid of alkali and/or alkaline-earth hydroxides, ammonia, an amine, polyamine or amino alcohol.

Preferred monomers of group a) are (meth)acrylamide and hydroxyalkyl(meth)-acrylate in which alkyl=C1 to C5, hydroxyalkyl-polyethyleneoxy- and -propylene-oxy and -butyleneoxy(meth)acrylate in which alkyl=C1 to C5.

For certain applications it may be advantageous when the water-soluble polymer has an average molecular weight of Mn of 1000 to 100,000 g/mol and preferably between 2000 and 70,000 g/mol, particularly preferably between 3000 and 50,000 g/mol which is also taken into consideration by the present invention.

With regard to the monomers b) it should be noted that in the case of polyethylene-oxyvinyl ethers, polypropyleneoxyvinyl ethers and the analogous polyethylene-oxyallyl ethers or polypropyleneoxyallyl ethers such as e.g. polyethylene glycol monoallyl ethers or mixed EO-PO-monoallyl ethers, the chain length of the polyalkyloxy building blocks of the monomers is in principle unlimited but n≤10 has certain advantages.

The water-soluble polymers that are used according to the invention can be obtained using a number of possible production processes. Preferred methods are radical polymerization in water or in an aqueous mixture containing a maximum of 30% by weight of an organic solvent, and also emulsion polymerization and solvent-free bulk polymerization.

The water-soluble polymers as drying aids can be used in a dry form e.g. in a powder form and also in the form of aqueous solutions that contain 1 to 99% by weight of the water-soluble polymers, amounts between 20 and 70% by weight being preferred and amounts between 35 and 60% by weight being especially preferred.

Irrespective of how the water-soluble polymers have been obtained or whether the drying aids are used in a powder form or in an aqueous solution, the present invention provides that the drying aids are added in the production process for dispersants or redispersion powders in amounts of up to 50% by weight and preferably between 5 and 30% by weight, in particular from 10 to 20% by weight based on the dispersant or the polymer dispersion.

Since dispersants are obtained in a powder form in particular with the aid of known spray drying processes, the present invention also takes into consideration the use of the drying aids preferably in these processes. In this case the drying aid can in particular be used in a contact drier, fluidized-bed drier or band drier or in a drying process that is carried out with the aid of heat radiation such as infrared or microwave radiation.

The water-soluble polymers can preferably be used in the production of a dispersant which is composed of structural elements containing polyoxyalkylene, carboxylic acid and/or carboxylic acid anhydride monomers as known in particular from the patent documents WO 97/39 037, EP-A 0 610 699 or WO 98/28 353.

With regard to the polymeric dispersant in powder form and the redispersion powder the invention provides that the drying aid is used in such a manner that a polymeric dispersant in powder form is obtained which has a residual moisture content of <5% by weight and preferably <2% by weight.

It may indeed be expedient to also add appropriate, more or less finely dispersed quality-improving additives to the polymeric dispersant that has been produced with the drying aid after its production so that the polymeric dispersant powder remains resistant to agglutination and caking during transport and storage in hot climate zones. There is no particular limitation to the type of these additives but the respective materials should have a good physical and chemical compatibility with the dispersant. The dispersive action of the agent should not be adversely affected and the quality-improving properties should be apparent even when only small amounts of the additives are added. In this connection the present invention envisages in particular the use of chalk, silicic acid, calcite, dolomite, quartz powder, bentonite, pumice powder, titanium dioxide, aluminium oxide, flue ash, cements, silicates, talcum, mica, anhydrite, lime, kieselguhr, gypsum, magnesite, clay, kaolin, powdered slate and powdered mineral stone, barium sulfate and mixtures thereof as quality-improving additives. The quality-improving additives should particularly preferably be used in a finely dispersed form and in this case in particular with a particle size of 0.1 to 1000 μm.

The chemical production network may include one or more one or more chemical and/or mechanical process(es). The chemical production network may produce one or more output material(s) through chemical and/or mechanical processing. The chemical production network may include multiple types of production processes for producing one or more output material(s) from one or more input material(s). The chemical production network may produce one or more output material(s) from input material(s) provided to the chemical production network. The chemical production network may include a complex production network producing multiple chemical products via multiple production process(es). The chemical production network may include connected, interconnected and/or non-connected production process(es). The chemical production network may include a composite or Verbund network.

The chemical production network may include identity preserving or segregated production process(es). Identity preserving or segregated in this context may refer to environmental attribute(s) of input material(s) being preserved or segregated in the production process(es). Examples are non-fossil, e.g. renewable or recycled, input materials used to produce the one or more Aqueous polymeric composition(s) without fossil content. Further examples are fossil input material(s) used to produce the one or more Aqueous polymeric composition(s) with fossil content. Chemical production networks may include non-identity preserving or non-segregated production process(es). Non-identity preserving or non-segregated in this context may refer to non-fossil input material(s) being mixed with fossil input material(s) to produce the aqueous polymeric composition(s). For example, fossil and renewable input materials may be mixed to produce the aqueous polymeric composition(s) with fossil and renewable content.

The chemical production network may include one or more production process(es) with multiple production steps. The production steps included in the chemical network may be defined by the physical system boundary of the chemical production network. The system boundary may be defined by location and/or control over production processes or steps. The system boundary may be defined by a site of the chemical production network. The system boundary may be defined by production process(es) or step(s) controlled by one entity or multiple entities jointly. The system boundary may be defined by the value chain with staggered production process(es) or step(s) to the chemical end product, which may be controlled by multiple entities jointly or separately. The chemical production network may include a waste collection, a sorting step, a recycling step such as pyrolysis, a cracking step such as steam cracking, a separation step to separate intermediates of one process step and further processing steps to convert such intermediates to output material(s), in particular Aqueous polymeric composition(s) leaving the system boundary of the chemical production network. The input material(s) may enter the physical system boundary of the chemical production network. The entry point(s) of the chemical production network may be marked by the entry of input material(s) to the chemical production network or the system boundary of the chemical network. The output material(s), in particular Aqueous polymeric composition(s) may leave the physical system boundary of the chemical production network. The exit point(s) of the chemical production network may be marked by the exit of output material(s), in particular Aqueous polymeric composition(s) from the chemical production network or the system boundary of the chemical network.

The chemical production network may include one or more production chain(s) for the production of Aqueous polymeric composition. The production chain(s) for the production of Aqueous polymeric composition may be interconnected. The production chain(s) for the production of Aqueous polymeric composition may be interconnected with production chain(s) for the production of other output material(s). The production chain(s) for the production of Aqueous polymeric composition may include production chain(s) for the production of intermediates used to produce Aqueous polymeric composition. The production chain(s) for the production of Aqueous polymeric composition may use input material(s) provided by chemical network(s) for the production of intermediates usable to produce Aqueous polymeric composition.

One or more input material(s) may be provided to the chemical production network for producing one or more output material(s), in particular Aqueous polymeric composition(s). The output material(s), in particular Aqueous polymeric composition(s) may be produced from one or more input material(s) through one or more chemical process(s) of the chemical production network. The input material may comprise any input material entering the chemical production network at any entry point. The input material may include an output material(s), in particular Aqueous polymeric composition(s) such as a natural, organic or inorganic output material(s), in particular Aqueous polymeric composition(s). The input material may be a pre-cursor product, an intermediate material, a feedstock or a raw material used to produce one or more output material(s), in particular Aqueous polymeric composition(s). The input material may be fed to the chemical production network to produce one or more output material(s), in particular Aqueous polymeric composition(s). The input material may be fed to chemical production network including one or more production process(es) with multiple process steps. The input material may be fed to the chemical production network at the start of the production process or at any intermediate stage of the production process. The input materials entering the chemical production network may be used to produce one or more output material(s), in particular Aqueous polymeric composition(s).

The input material may be associated with an input material identifier. The input material identifier may comprise any identifier uniquely associated with the input material. The input material identifier may be associated with the physical entity of the input material. The input material identifier may be associated with a single batch of input material. The input material identifier may be associated with a group of input materials. The identifier may be associated with multiple physical entities of the input material. The input material identifier may be associated with continuous or semi-continuous stream of input material. The input material identifier may be associated with a stream of the input material e.g. over a certain time period or from a certain supplier. The input material identifier may be linked or connected to one or more environmental attribute(s).

Environmental attribute may refer to a property related to the environmental impact. Such property may be the property of input material(s), chemical process(es), chemical production network(s) and/or Aqueous polymeric composition(s). The environmental attribute may indicate an environmental performance of input material(s), chemical process(es), chemical production network(s) and/or Aqueous polymeric composition(s). The environmental attribute may be derived from properties of input material(s), chemical process(es), chemical production network(s) and/or Aqueous polymeric composition(s). The environmental attribute may be associated with the environmental impact of input material(s), chemical process(es), chemical production network(s) and/or Aqueous polymeric composition(s) at any stage of the lifecycle of the aqueous polymeric composition(s). The stages may include providing raw material, providing feedstock, producing chemical products, such as intermediate products or end products, producing discrete products by using the chemical products, using chemical products or discrete products, treating end-of-life products, recycling end-of-life products, disposing end-of-life products, reusing components from end-of-life products or any subset of stages. The environmental attribute may be specified or may be derived from any activity of one or more entities participating at any stage of the lifecycle of one or more material(s) or product(s).

The environmental attribute may include one or more characteristic(s) that are attributable to environmental impact of input material(s), chemical process(es), chemical production network(s) and/or Aqueous polymeric composition(s). The environmental attribute may include environmental, technical, recyclability or circularity characteristics(s) associated with the environmental impact of input material(s), chemical process(es), chemical production network(s) and/or Aqueous polymeric composition(s).

The environmental attribute may include one or more characteristic(s) that are attributable to the environmental impact of input material(s), chemical process(es), chemical production network(s) and/or Aqueous polymeric composition(s). The environmental attribute may include environmental, technical, recyclability, safety, legal, or circularity characteristics(s), preferably environmental, technical, recyclability, circularity characteristics(s) associated with the environmental impact of input material(s), chemical process(es), chemical production network(s) and/or Aqueous polymeric composition(s). The one or more environmental attribute(s) may be attributable to the environmental impact of the aqueous polymeric composition. The one or more environmental attribute(s) may relate to environmental, technical, recyclability, circularity and/or complementary risk characteristic(s) of the aqueous polymeric composition.

Environmental characteristic(s) may specify or quantify ecological or compositional criteria associated with environmental impact. Environmental characteristic(s) may be or may be derived from measurements taken during the lifecycle. Environmental characteristics may be determined at any stage of the lifecycle and may characterize the environmental impact for such stage or up to such stage. Environmental characteristic(s) may for example include carbon footprint, greenhouse gas emissions, resource usage, air emissions, ozone depletion potential, water pollution, noise pollution, energy consumption, waste reduction, or eutrophication potential. Environmental characteristic(s) may for example include product characteristics like compositions, impurity profiles, or information on substances of concern as defined by industry specific regulatory or value chain requirements or product characteristics related to the production of the product like bio based, vegetable based, animal based, halogen-free, fluorine-free, vegan, halal, kosher, palm oil-free, natural, free from hazardous compounds, volatile organic compounds-free or any combinations thereof. Preferably environmental characteristic(s) include product characteristics related to the production of the product like bio based, vegetable based, animal based, halogen-free, fluorine-free, vegan, halal, kosher, palm oil-free, natural, free from hazardous compounds, volatile organic compounds-free or any combinations thereof.

Technical characteristic(s) may specify or quantify performance at least indirectly associated with the environmental impact. Technical characteristic(s) may be or may be derived from measurements taken during the lifecycle. Technical characteristics may be determined at any stage of the lifecycle and may characterize the performance for such stage or up to such stage. Technical characteristic(s) may for example include chemical composition data, raw material composition such as bio-based or recycled input material content specifying e.g. x % non fossil and y % fossil content, bill of materials, product specification data such as product purity, product form (as indication to their impact on dust formation/release), safety data, product extractability, migration data, toxicological data or ecotoxicological data, product component data, safety data, application property data, application instructions, quality data or any combinations thereof.

Circularity characteristic(s) may specify or quantify the life cycle characteristics associated with circular uses. Circularity characteristic(s) may be or may be derived from measurements taken during the lifecycle. Circularity characteristic(s) may be or may be derived from circular data recorded in one or more prior lifecycle(s) including reuse. Circularity characteristic(s) may be determined at any stage of the lifecycle and may characterize the reuse or recycling performance for such stage or up to such stage. Circularity characteristic(s) may for example include recycling data, reuse rate, recycling rate, recycling loops, reuse performance, reused quality or any combinations thereof.

Recyclability characteristic(s) may specify or quantify life cycle characteristics associated with recycling uses. Recyclability characteristic(s) may include the composition of the material including specifically tailored constituents making the material suitable for recycling. Recyclability characteristic(s) may be or may be derived from measurements taken during the lifecycle. Recyclability characteristic(s) may be or may be derived from recycling data recorded in one or more prior lifecycle(s). Recyclability characteristics may be determined at any stage of the lifecycle and may characterize the recycling performance for such stage or up to such stage. Recyclability characteristic(s) may for example include recycling data, number of reuses, recyclate composition, recyclate quality, waste stream composition, waste stream quality or any combinations thereof.

Environmental impact may for example include impacts on the safe use and application and compliance with mandatory and/or voluntary assessment schemes needed for the application or required by the value chain in which the product itself, formulation of said product and conversion products of said products are handled.

In one embodiment the aqueous polymeric composition passport or digital asset of the aqueous polymeric composition may include mass balanced environmental attributes related to the input material. Mass balanced environmental attributes may include environmental attributes of the input material(s) used to produce the aqueous polymeric composition, which are tracked and by mass attributable to the aqueous polymeric composition. The environmental impact of input material(s) may be determined based on input material(s) used in the chemical process(s) to produce the aqueous polymeric composition. For example, bio-based, renewable and/or recycled content of input material(s) used to produce the aqueous polymeric composition may be tracked. Further for example, castor oil or palm oil content of input material(s) used to produce the aqueous polymeric composition may be tracked. Further for example, properties related to the environmental impact of the input material include RSPO palm oil, palm oil free or castor oil may be tracked. Further for example, properties of the chemical process(es) used to produce the aqueous polymeric composition may be tracked. Examples of tracked process properties related to the environmental impact include water consumption, CO₂ emissions and/or Greenhouse Gas (GHG) emissions, amount of waste generation, mixed material generation, design for recycling, energy consumption, processing properties such as less waste or less loss of properties. The properties may be tracked based on a certificate from a certifying agency. The properties may be tracked based on inherent physical properties derived from measurements.

In one embodiment the produced Aqueous polymeric composition is connected to the decentral identifier physically identifying the produced Aqueous polymeric composition. The production operating apparatus may be configured to provide the decentral identifier associated with a physical entity of the produced Aqueous polymeric composition. The production operating apparatus may be configured to link the decentral identifier to a physical identifier of the produced Aqueous polymeric composition. The production operating apparatus may be configured to assign the decentral identifier to the physical identifier connected to the produced Aqueous polymeric composition.

In one embodiment the decentral identifier relates to data associated with at least one product produced from the aqueous polymeric composition, wherein the one or more environmental attribute(s) associated with the at least one product is derived from one or more environmental attribute(s) associated with the aqueous polymeric composition. The one or more environmental attribute(s) associated with the aqueous polymeric composition may be associated with the one or more input material(s) and/or the chemical process(s) used to produce the aqueous polymeric composition. The decentral identifier may relate to any identifier uniquely associated with the aqueous polymeric composition. The decentral identifier may be associated with the physical entity of the aqueous polymeric composition. The decentral identifier may refer to a single batch of Aqueous polymeric composition. The decentral identifier may be associated with a group of Aqueous polymeric composition. The identifier may refer to multiple physical entities of the aqueous polymeric composition. The decentral identifier may be associated with continuous or semi-continuous stream of Aqueous polymeric composition. The identifier may refer to a stream of the aqueous polymeric composition e.g. over a certain time period or from a certain supplier.

In one embodiment the one or more environmental attribute(s) associated with the aqueous polymeric composition(s) are provided from at least one balancing account configured to store environmental attribute(s) associated with input material(s). An inbound allocator may be configured to allocate the one or more environmental attribute(s) associated with input material(s) to at least one balancing account e.g. on entry of the input material to the chemical production network. The balancing account may be associated with the respective environmental attribute type. An outbound assigner may be configured to assign at least one environmental attribute from the at least one balancing account associated with the respective environmental attribute to the decentral identifier. One or more environmental attribute(s) may be assigned to the at least one decentral identifier. Assignment may include de-allocation of the one or more environmental attributes from the balancing account associated with the respective environmental attribute type. By using the balancing accounts environmental attributes of input materials can be reliably tracked and assigned to Aqueous polymeric composition.

In one embodiment the one or more environmental attribute(s) associated with the aqueous polymeric composition(s) are provided from at least one balancing account configured to store environmental attribute(s) associated with input material(s). An inbound allocator may be configured to allocate the one or more environmental attribute(s) to at least one balancing account associated with the respective environmental attribute, e.g. on input material entering the chemical production network. The balancing account may be associated with the respective environmental attribute type. The one or more environmental attribute(s) associated with the input materials may be allocated to the balancing account associated with the respective environmental attribute type. An outbound assigner may be configured to assign or link at least one environmental attribute from the at least one balancing account associated with the respective environmental attribute type to the decentral identifier. This may include de-allocation of the one or more environmental attribute(es) from the balancing account associated with the respective environmental attribute type. By using the balancing accounts environmental attributes can be tacked through the chemical production network. This way the environmental attributes may be detached from the material flow. Attribution of environmental attribute(s) may be conducted on a mass balance basis for the chemical production network. In such approach the total mass of input materials and Aqueous polymeric composition as well as the attribution of respective environmental attribute(s) associated with input materials and Aqueous polymeric compositions are balanced.

In one embodiment the one or more environmental attribute(s) are associated with at least one property related to the environmental impact of the one or more input material(s) and/or the chemical process(s). The one or more environmental attribute(s) may specify environmental properties of the input material(s) used to produce the aqueous polymeric composition and/or the one or more environmental attribute(s) may specify environmental properties of the chemical process(es) used to produce the aqueous polymeric composition. The one or more environmental attribute(s) may be generated from environmental properties of the input material(s) used to produce the aqueous polymeric composition, process data associated with the chemical processing of the input material(s) and/or energy data associated with the energy consumption of the chemical processing. The one or more environmental attribute(s) may include a recycled content associated with the input material(s) and allocated or allocatable to the aqueous polymeric composition(s), a renewable content associated with the input material(s) and allocated or allocatable to the aqueous polymeric composition(s), and/or a product carbon footprint associated with the aqueous polymeric composition(s).

In one embodiment the production operating apparatus is configured to gather environmental attributes associated with the produced Aqueous polymeric composition before, during and/or after production of the aqueous polymeric composition by the chemical production network. The environmental attributes associated with the produced Aqueous polymeric composition may relate to input material(s). The environmental attributes associated with input materials may be provided before, during and/or after production of the aqueous polymeric composition by the chemical production network. The environmental attributes associated with input materials may be allocated to at least one balancing account before, during and/or after production of the aqueous polymeric composition by the chemical production network. The environmental attributes associated with the produced Aqueous polymeric composition may relate to environmental properties generated from process data associated with the chemical processing of the input material(s) and/or energy data associated with the energy consumption of the chemical processing. The environmental attributes associated with the produced Aqueous polymeric composition may be generated before, during and/or after production of the aqueous polymeric composition by the chemical production network. The process data associated with the chemical processing of the input material(s) and/or energy data associated with the energy consumption of the chemical processing may be gathered prior, during and/or after production of the aqueous polymeric composition.

In one embodiment the aqueous polymeric composition passport or the digital asset include the decentral identifier associated with the aqueous polymeric composition and the one or more environmental attribute(s) linked to the decentral identifier. The one or more environmental attribute(s) may be linked to the decentral identifier included the aqueous polymeric composition passport or the digital asset. The one or more environmental attribute(s) may be stored in a data base of the aqueous polymeric composition producer for access by any Aqueous polymeric composition user. The one or more environmental attribute(s) may be stored in a data base of the aqueous polymeric composition producer for transfer to an aqueous polymeric composition user e.g. when accessed or on providing the aqueous polymeric composition. The decentral identifier may comprise any unique identifier uniquely associated with the aqueous polymeric composition producer and Aqueous polymeric composition data such as the environmental attributes. The decentral identifier may include a Universally Unique IDentifier (UUID) or a Digital IDentifier (DID). The decentral identifier may be issued by a central or decentral identity issuer. The decentral identifier may be linked to authentication and/or authorization information. Via the decentral identifier and its unique association with the aqueous polymeric composition producer and Aqueous polymeric composition data, such as the environmental attributes, access to the aqueous polymeric composition data may be controlled by the aqueous polymeric composition producer. This contrasts with central authority schemes, where identifiers are provided by such central authority and access to data is controlled by such central authority. Decentral in this context refers to the usage of the identifier in implementation as controlled by the data owner, such as the aqueous polymeric composition producer.

The decentral identifier may be uniquely associated with the aqueous polymeric composition or the physical entity of the aqueous polymeric composition, e.g. as packaged for transportation to the aqueous polymeric composition user. The decentral identifier may be uniquely to the one or more environmental attribute(s). The Aqueous polymeric composition passport or the digital asset may include one or more digital representation(s) pointing to Aqueous polymeric composition data including the environmental attribute(s) or parts thereof including the environmental attribute(s). The digital representation may comprise at least one interface to a data providing service. It may further include at least one interface to a data consuming service. It may include an endpoint for data exchange or sharing (resource endpoint) or an endpoint for service interaction (service endpoint), that is uniquely identified via a communication protocol. The digital representation(s) pointing to Aqueous polymeric composition data or parts thereof may be uniquely associated with the decentral identifier.

The Aqueous polymeric composition passport or the digital asset may comprise or be connected to a digital representation of Aqueous polymeric composition data, such as environmental attribute(s). The digital representation may include a representation for accessing the aqueous polymeric composition data, such as environmental attribute(s) or part thereof. The digital representation may include a representation of Aqueous polymeric composition data, such as environmental attribute(s). The Aqueous polymeric composition passport or the digital asset may include or be connected to data related to the aqueous polymeric composition data, such as environmental attribute(s), the authentication information and the decentral identifier. The data related to the aqueous polymeric composition data, such as environmental attribute(s) may include the digital representation of the aqueous polymeric composition data, such as environmental attribute(s).

### BRIEF DESCRIPTION OF THE DRAWINGS

In the following, the present disclosure is further described with reference to the enclosed figures:

FIG. 1 illustrates schematically an example of a chemical production network producing one or more output material(s) from one or more input material(s) in connection with a production operating system including an attribute management system.

FIG. 2 illustrates schematically an example of attributing environmental attributes of input materials to output materials of the chemical production network.

FIG. 3 illustrates schematically an example of attributing environmental attributes of input materials and chemical processes to an output material of the chemical production network.

FIG. 4 illustrates schematically another example of a method or apparatus for providing environmental attributes associated with output materials to a material user as data consumer via a decentral network.

FIG. 5 illustrates schematically an example of a method or apparatus for providing environmental attributes of output materials across value chains via the decentral network.

FIG. 6 illustrates schematically an example of a chemical production network for producing Aqueous polymeric composition associated with the digital asset.

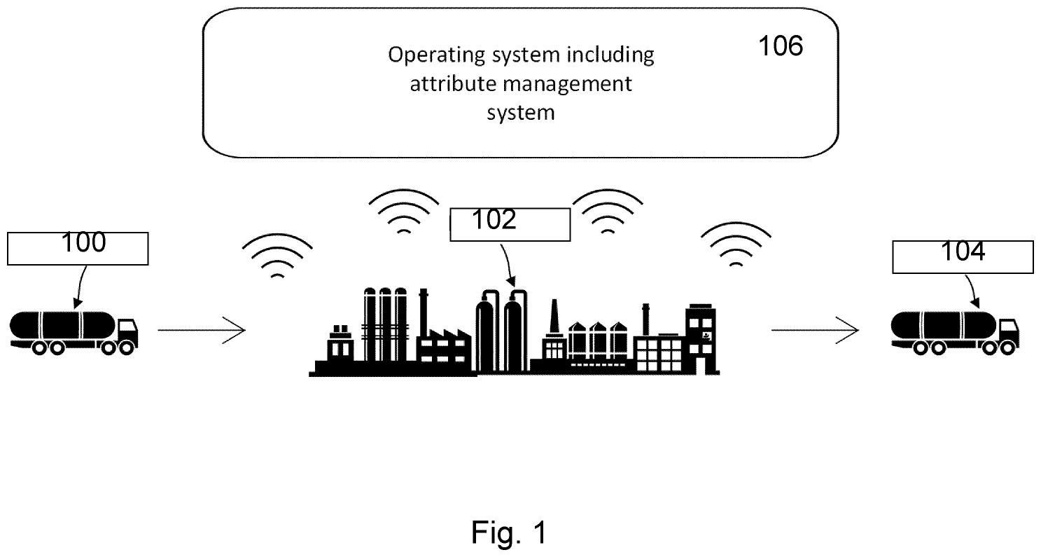

FIG. 1 illustrates an example of a chemical production network 102 producing one or more output material, in particular Aqueous polymeric composition(s) 104 from one or more input material(s) 100 in connection with a production operating system 106 including an attribute management system.

For producing one or more output material(s), in particular Aqueous polymeric composition(s) 104 different input materials 100 may be provided as physical inputs to the chemical production network 102. The physical input and output material(s), in particular Aqueous polymeric composition(s) 100, 104 may be associated with one or more properties related to environmental impact. The properties related to environmental impact may be digitalized in the form of environmental attributes such as recycled or bio-based content of the input materials. The production operating system 106 may be configured to ingest such environmental attributes and to track the environmental attributes across the chemical production network 102 from input materials 100 to output material(s), in particular Aqueous polymeric composition(s) 104.

The chemical production network 102 may include multiple interlinked processing steps. The chemical production network 102 may be an integrated chemical production network 102 with interrelated production chains. The chemical production network 102 may include multiple different production chains that have at least one intermediate product in common. The chemical production network 102 may include multiple stages of the chemical value chain. The chemical production network 102 may include the producing, refining, processing and/or purification of gas or crude oil. The chemical production network 102 may include a stream cracker, or a syngas plant connected to multiple production chains that output chemical products 104 from the effluent of such plants. The chemical production network 102 may include multiple production chains that output from one or more input material(s) 100 one or more output material(s), in particular Aqueous polymeric composition(s). The chemical production network 102 may include multiple tiers of a chemical value chain. The chemical production network 102 may include a physically interconnected arrangement of production sites. The production sites may be at the same location or at different locations. In the latter case the production sites may be interconnected by means of dedicated transportation systems such as pipelines, supply chain vehicles, like trucks, supply chain ships or other cargo transportation means.

The chemical production network 102 may chemically convert input materials 100 to one or more output material(s), in particular Aqueous polymeric composition(s) 104. The chemical production network 102 may convert input materials 100 by way of chemical conversion to one or more output material(s), in particular Aqueous polymeric composition(s) 104.

The input materials 100 may be fed to the chemical production network 102 at any entry point. The input materials 100 may be fed to the chemical production network 102 at the start of the chemical production network 102. Input materials 100 may for example make up the feedstock of a steam cracker. The input material 100 may include non-fossil input material, such as biobased or recycled material, and/or fossil input material for the manufacture of chemical intermediates and chemical output material(s), in particular Aqueous polymeric composition(s) 104.

The chemical production network 102 may include multiple production steps. The production steps included in the chemical production network 102 may be defined by the system boundary of the chemical production network 102. The system boundary may be defined by location or control over production processes. The system boundary may be defined by the site of the chemical production network 102. The system boundary may be defined by production processes controlled by one entity or multiple entities jointly. The system boundary may be defined by value chain with staggered production processes to an end product, which may be controlled by multiple entities separately. The chemical production network 102 may include a waste collection and sorting step, a recycling step such as pyrolysis, a cracking step such as steam cracking, a separation step to separate intermediates of one process step and further processing steps to convert such intermediates to output material(s), in particular Aqueous polymeric composition(s) 104 leaving the system boundary of the chemical production network 102.

The production operating system 106 of the chemical production network 102 may be configured to monitor and/or control the chemical production network 102 based on operating parameters of the different processes. One process step monitored and/or controlled may be the feed of input materials 100 or the release of output material(s), in particular Aqueous polymeric composition(s) 104. Another process step monitored and/or controlled may be the registration of environmental attributes associated with input materials 100 entering the system boundary of the chemical production network 102. Yet another process step monitored and/or controlled may be the attribution of environmental attributes to output material(s), in particular Aqueous polymeric composition(s) 104 produced via the chemical production network. Yet another process step monitored and/or controlled may be the management of environmental attributes associated with input materials 100 and output material(s), in particular Aqueous polymeric composition(s) 104 of the chemical production network 102.

The production operating system 106 may be configured to register inbound environmental attributes, to assign outbound environmental attributes. The production operating system 106 may be configured to access data related the inputs materials 100, the processes and/or the output material(s), in particular Aqueous polymeric composition(s) 104 of the chemical production network 102. For example, the production operating system 106 may be configured to register a recycled or bio-based content of the one or more input material(s) 100 used in the chemical production network 102 as environmental attribute. The production operating system 106 may be configured to allocate the environmental attribute to at least one balancing account associated with the recycled or bio-based content of the input materials 100. The production operating system 106 may be configured to allocate at least a part of the environmental attributes from the at least one balancing account to the at least one output material(s), in particular Aqueous polymeric composition(s) 104.

The production operating system 102 may be configured to handle environmental attributes related to the input and output material(s), in particular Aqueous polymeric composition(s) 100/104 of the chemical production network 102. For example, the production operating system 106 may be configured to determine environmental attributes associated with the use of input materials 100 impacting the environmental property of the chemical production network 102 and the output material(s), in particular Aqueous polymeric composition(s) 104 produced by the chemical production network 102. Further in particular, the production operating system 102 may be configured to determine environmental attributes associated with the output material(s), in particular Aqueous polymeric composition(s) 104. This way the production operating system 102 may be configured to store environmental attributes in balancing accounts or to delete environmental attributes from the balancing accounts. The environmental attributes may hence be viewed as a credit that may be deposited in an account or deducted from an account related to the input and output material(s), in particular Aqueous polymeric composition(s) of the chemical production network 102. This way the environmental impact of the production may be tracked and/or traced.

In chemical production networks 102 multiple value chains may be linked. Additionally different input materials 100 or chemical processes impacting the environmental property of output material(s), in particular Aqueous polymeric composition(s) 104 produced by the chemical production network 102 may be used. Examples of input materials 100 impacting at least one environmental property of output material(s), in particular Aqueous polymeric composition(s) 104 produced from such input materials 100 are recycled, renewable or bio-based input materials 104. Examples of chemical processes impacting the environmental property include chemical processes using environmentally friendly technology such as carbon capture, carbon utilization or heat pumps.

Owing to the processing of chemicals in continuous or semi-continuous production and the complexity of chemical production networks 102, traceability of the input materials through the network may be hampered. In such scenarios, an equivalent environmental attribute signifying the impact on the environmental property of output material(s), in particular Aqueous polymeric composition(s) 104 produced by the chemical production network may be allocated to balancing accounts 122 and assigned to one or more output material(s), in particular Aqueous polymeric composition(s) 104 of the chemical production network 102. The environmental attributes may hence be decoupled from the physical material flow inside the chemical production network 102. Decoupling may be based on the mass balance model in that the equivalent amount assigned to the one or more output material(s), in particular Aqueous polymeric composition(s) may not exceed the equivalent amount provided by input materials or processes. If an equivalent amount has been allocated to the virtual account of one environmental attribute type, it may not be allocated a second time to another virtual account of the one environmental attribute type. Environmental attribute types may be recycled, bio-based, renewable or the like. Environmental attributes may be provided in the form of digital assets or Aqueous polymeric composition passports attached to the physical entity of the aqueous polymeric composition.

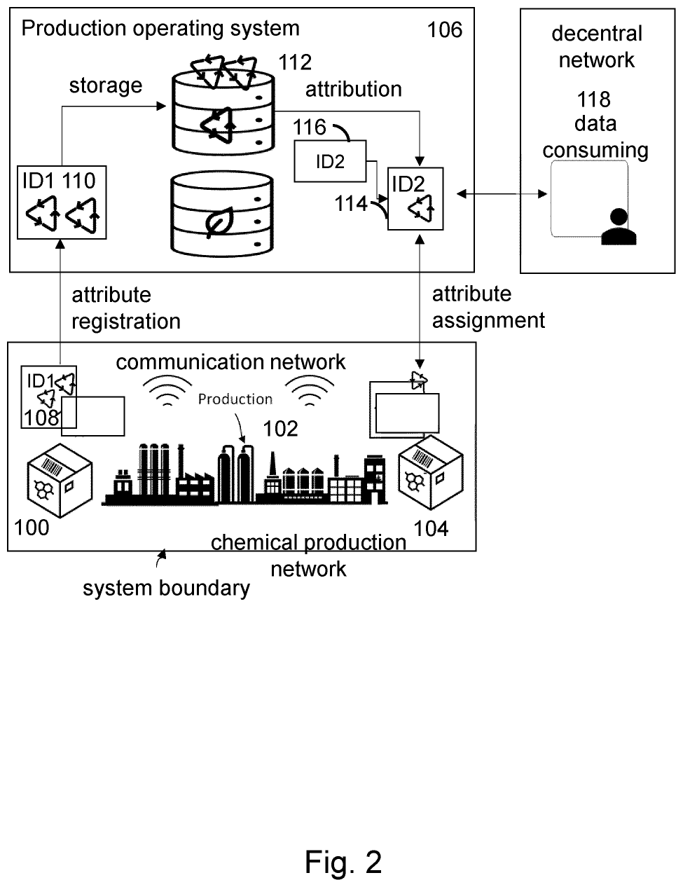

FIG. 2 illustrates schematically an example of attributing environmental attributes associated with input materials 100 to output material(s), in particular Aqueous polymeric composition(s) 104 of the chemical production network 102.

As shown in FIG. 1 the chemical production network 102 and operations of the chemical production network 102 may be monitored and/or controlled by a production operating system 106. The production operating system 106 may be configured to track environmental attributes from input materials 100 fed to the chemical production network 102 to output material(s), in particular Aqueous polymeric composition(s) 104 produced by the chemical production network 102. For tracking the operating system 106 may be configured to register environmental attributes associated with the input materials 100 provided to the chemical production network 102 and to attribute environmental attributes to output material(s), in particular Aqueous polymeric composition(s) 104 produced by the chemical production network 102.

The input materials 100 such as pyrolysis oil, bio-naphtha or bio-gas may be provided to the chemical production network 102. The input materials 100 may enter the chemical production network at the entry point, such as a such as a steam cracker or a syngas plant. The input materials 100 may be used in the chemical production network 102 to produce one or more output material(s), in particular Aqueous polymeric composition(s) 104 from the input materials 100. output material(s), in particular Aqueous polymeric composition(s) 104 may be provided at exit points of the chemical production network 102.

On entry of the input material 100, input material data 108 may be provided via a communication network to a computing interface of the production operating system 106. A data provider, such as a QR code reader, may be configured to provide material data 108 related to the one or more input material(s) 100 and respective environmental attributes 108 to a computing interface configured to allocate the environmental attributes associated with the input materials 100. The material data 108 may include the input material identifier and environmental attributes associated with the input materials 100. The input material identifier may be associated with the physical entity of the input material 100 entering the chemical production network 102. The material data may be provided on, prior or after providing of the one or more input material(s) at entry points to the chemical production network 102.

The input material identifier may be linked to the environmental attribute(s) associated with the respective input material(s) 100, the amount of input material 100 and the certificate certifying the environmental attribute(s). The amount of input material may be a measured amount of input material 100 fed to a plant or storage of the chemical production network 102 for producing one or more output material(s), in particular Aqueous polymeric composition(s) 104 from the input material(s) 100. The input material identifier associated with the respective input material 100, the environmental attribute(s) associated with the respective input material(s) 100 and the amount of input material(s) 100 provided to the chemical production network 102 may be provided to the production operating system 106. Such data may be provided via a communication network on entry to chemical production network 102, or the data may be transferred from a computing system to the production operating system 106.

An inbound allocator 110 may be configured to allocate the one or more environmental attribute(s) to at least one balancing account 112 associated with the respective environmental attribute. For example, one balancing account 112 may relate to environmental attributes from recycled material and another balancing account 112 may relate to environmental attributes from bio-based material. The balancing account may be associated with the respective environmental attribute type, such as bio-based or recycled 100. Based on such association the balancing account associated with the environmental attribute type of the respective input material 100 may be selected. The environmental attributes may be allocated to the selected balancing account. For example, the account 112 for recycled material may be selected and the environmental attribute may be allocated to such account 122.

To allocate, the one or more environmental attribute(s) may be converted to balancing units and the balancing units may be allocated to the balancing account 122. The conversion may be based on a conversion factor such as mass, weight, carbon atoms, hydrogen atoms, methane equivalents or any other suitable measure for quantifying the environmental impact of the environmental attribute. The conversion factor may hence take into account the difference between producing chemical products from conventional input material(s) and producing chemical products from non-conventional input material(s) or producing chemical products from a mix of conventional and non-conventional input materials. The conversion factor may relate to differences in chemical and/or physical properties of conventional and non-conventional input material(s).

By using the balancing accounts 112 it can be ensured that environmental attributes of input materials 100 are only used once for assignment to Aqueous polymeric composition 104. This way double counting on input or output is avoided to ensure positive environmental impact can be reliable tracked and assigned to Aqueous polymeric composition 104.

An identifier provider 116 may be configured to provide the aqueous polymeric composition identifier associated with the aqueous polymeric composition produced by the chemical production network 102 and provided at the exit point from the chemical production network 102.

An outbound assigner 114 may be configured to assign at least one environmental attribute from the at least one balancing account 112 associated with the respective environmental attribute to the aqueous polymeric composition identifier ID2. One or more environmental attribute(s) may be assigned to the at least one Aqueous polymeric composition identifier ID2. Assignment may include de-allocation of the one or more environmental attributes from the balancing account 112 associated with the respective environmental attribute type. Assignment may include converting one or more balancing unit(s) to one or more environmental attribute(s).

Assigning at least one environmental attribute associated with input material(s) to Aqueous polymeric composition(s) may include the linking of the aqueous polymeric composition identifier ID2 with the environmental attribute. The Aqueous polymeric composition identifier ID2 may be associated with the physical entity of the aqueous polymeric composition. This way the virtual identifier of a material may be uniquely linked to the physical material. Such linking may include a physical or virtual link of identifiers uniquely associated with the physical material. For physical linking a tag or code may be physically connected to the material, e.g., by printing a QR code on the packaging. For virtual linking different identifiers associated with the physical material may be linked. For example, an order number, a batch number, LOT number or a combination thereof may be linked.

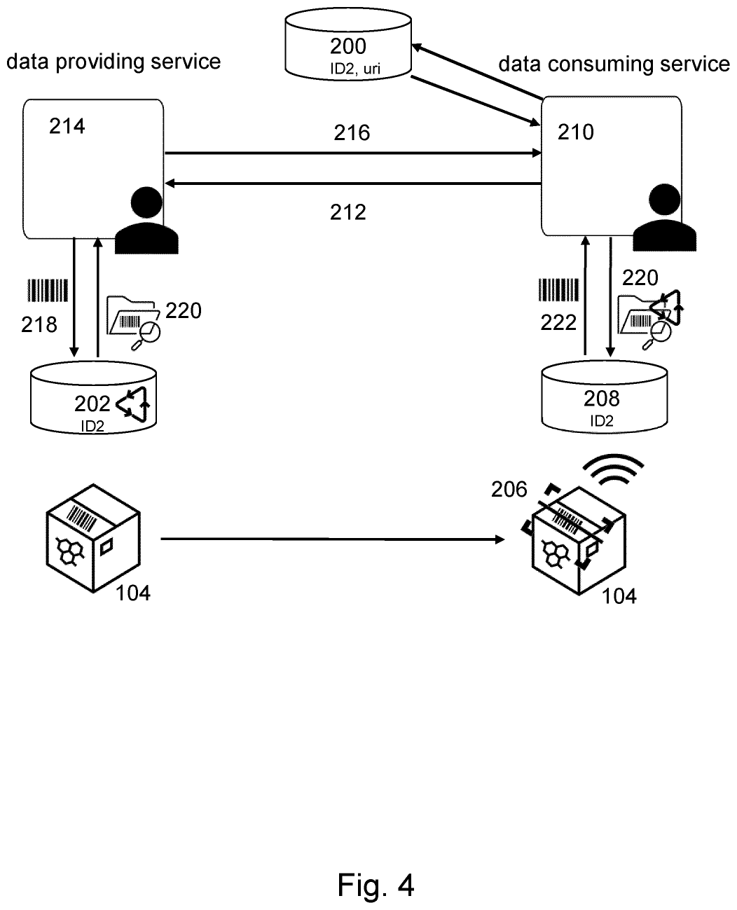

The outbound assigner 114 may be configured to provide the environmental attributes associated with the aqueous polymeric composition to a data consumer, such as a system associated with a user of the aqueous polymeric composition. The outbound assigner 114 may be configured to provide the environmental attributes associated with the aqueous polymeric composition to a decentral network as will be described in the example of FIG. 4. Environmental attributes may be provided via the above ID based schema in the form of digital assets or Aqueous polymeric composition passports associated with the physical entity of the aqueous polymeric composition.

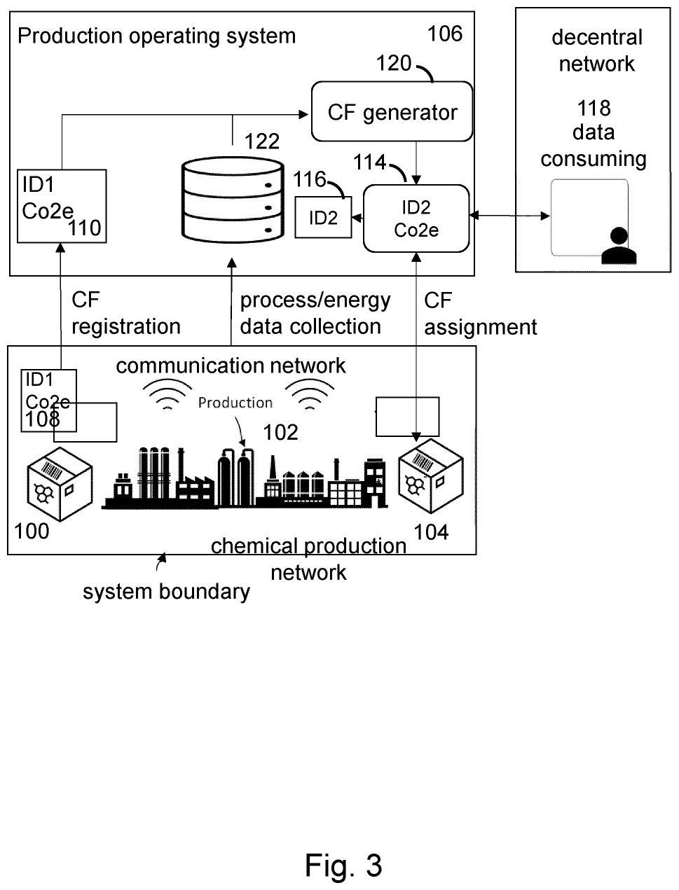

FIG. 3 illustrates schematically an example of attributing environmental attributes of input materials 100 and chemical processes to the aqueous polymeric composition 104 of the chemical production network 102.

As described in the context of FIGS. 1 and 2 the chemical production network 102 and operations of the chemical production network 102 may be monitored and/or controlled by a production operating system 106. Input materials 100 such as pyrolysis oil, bio-naphtha or bio-gas may be provided to the chemical production network 102. The input materials 100 may be used in the chemical production network 102 to produce one or more Aqueous polymeric composition(s) 104 from the input materials 100.

On entry of the input material 100, input material data 108 may be provided via a communication network to a computing interface of the production operating system 106. A data provider, such as a QR code reader, may be configured to provide material data 108 related to the one or more input material(s) 100 and respective environmental attributes 108 to a computing interface configured to allocate the environmental attributes associated with the input materials 100. The material data 108 may include the input material identifier and environmental attributes associated with the input materials 100. The input material identifier may be associated with the physical entity of the input material 100 entering the chemical production network 102. The input material identifier may be linked to the carbon footprint of the input material 100 as environmental attribute. The material data may be provided on, prior or after providing of the one or more input material(s) at entry points to the chemical production network 102.

The inbound allocator 110 may be configured to retrieve the one or more environmental attribute(s) and to provide such attributes to the carbon footprint (CF) generator 120. A process data provider 122 may be configured to gather process data associated with the chemical processing of the input material(s) 100 to produce the aqueous polymeric composition(s) 104. The process data provider 122 may be configured to gather energy data associated with the energy consumption of the chemical processing. The process data provider 122 may be configured to provide the process data and the energy data to the CF generator 120.

The CF generator 120 may be configured to determine the carbon footprint of the aqueous polymeric composition produced by the chemical production network. The carbon footprint of the of the aqueous polymeric composition may be determined based on the process data, the energy data and the carbon footprint of the input material(s) 100 used to produce the aqueous polymeric composition.

An identifier provider 116 may be configured to provide the aqueous polymeric composition identifier associated with the aqueous polymeric composition produced by the chemical production network 102 and provided at the exit point from the chemical production network 102.

An outbound assigner 114 may be configured to assign the determined carbon footprint to the aqueous polymeric composition identifier ID2. One or more environmental attribute(s) may be assigned to the at least one Aqueous polymeric composition identifier ID2, such as described in the context of FIG. 2.

The outbound assigner 114 may be configured to provide the environmental attributes, in particular the carbon footprint, associated with the aqueous polymeric composition to a data consumer, such as a system associated with a user of the aqueous polymeric composition. The outbound assigner 114 may be configured to provide the environmental attributes associated with the aqueous polymeric composition to a decentral network as will be described in the example of FIG. 4. Environmental attributes may be provided via the above ID based schema in the form of digital assets or Aqueous polymeric composition passports associated with the physical entity of the aqueous polymeric composition.

FIG. 4 illustrates schematically an example of a method or apparatus for providing environmental attributes associated with Aqueous polymeric composition to a material user as data consumer via a decentral network.

The Aqueous polymeric composition 104 as produced by the chemical production network 102 may be provided in association with the digital asset as described in the context of FIGS. 2 and 3. The digital asset may include the aqueous polymeric composition identifier. The digital asset may include one or more environmental attribute(s) such as the product carbon footprint, recycled content or bio-based content. The digital asset may relate to one or more environmental attribute(s) such as the product carbon footprint, recycled content or bio-based content. The digital asset may include a digital representation of one or more environmental attribute(s) such as the product carbon footprint, recycled content or bio-based content.

The digital asset may further include or relate to authentication and/or authorization information linked to the aqueous polymeric composition identifier. The authentication and/or authorization information may be provided for authentication and/or authorization of a data provider 208 and/or data consumer 210. The Aqueous polymeric composition identifier may include or relate to a decentral identifier, that is uniquely associated with the aqueous polymeric composition. The decentral identifier may be connected to the digital representation of the environmental attributes. The digital representation may include a representation for accessing the environmental attributes or parts thereof. The decentral identifier may include a Universally Unique IDentifier (UUID) or a Digital IDentifier (DID). The decentral identifier may include any unique identifier uniquely associated with a data owner and/or Aqueous polymeric composition. The data owner may be the producer of the aqueous polymeric composition. Via the decentral identifier and its unique association with the data owner and/or Aqueous polymeric composition access to the material configuration data may be controlled by the data owner.

The digital asset including the digital representation of one or more environmental attribute(s) such as the product carbon footprint, recycled content or bio-based content may be stored in a decentral data base 200. The one or more environmental attribute(s) such as the product carbon footprint, recycled content or bio-based content may be stored in a data base 202 associated with the data owner, such as the producer of the aqueous polymeric composition 104.

The Aqueous polymeric composition 104 may be physically delivered to a user of the aqueous polymeric composition. The Aqueous polymeric composition may be connected with a QR-code having encoded the aqueous polymeric composition identifier. The user of the aqueous polymeric composition may read the QR-code through a QR-code reader 206. The Aqueous polymeric composition identifier may be provided to a data base 208 associated with the user of the aqueous polymeric composition 104. In other embodiments the user of the aqueous polymeric composition may retrieve the aqueous polymeric composition identifier through the decentral data base 200.

The data owner in this example may be the input material producer, the output material producer, the output material user, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service may comprise computer-executable instructions for accessing and/or processing data, such as aqueous polymeric composition data, associated with the data owner. The data providing service may comprise computer-executable instructions for providing and/or processing data, such as Aqueous polymeric composition data, associated with the data owner for accessing and/or processing by the data consuming service.

Based on the received Aqueous polymeric composition identifier a request to access the environmental attributes associated with the aqueous polymeric composition identifier may be triggered by the data consuming service 210 as signified by arrow 212. The Aqueous polymeric composition identifier may be provided to the data providing service 214 of the producer of the aqueous polymeric composition 104. In addition, authentication and/or authorization information may be provided.

The request may be authenticated and/or authorized to access the environmental attributes associated with the aqueous polymeric composition identifier. Based on successful authorization and/or authentication access to the environmental attributes associated with the aqueous polymeric composition identifier may be granted.

For access the aqueous polymeric composition identifier may be provided to the data providing service 214 as signified by arrow 212. The data providing service 214 may use the received Aqueous polymeric composition identifier to retrieve the environmental attributes associated with the aqueous polymeric composition 104 as signified by arrows 218 and 220. The environmental attributes associated with the aqueous polymeric composition 104 provided to the data providing service 214 may be provided to the data consuming service 210 as signified by arrow 216. The environmental attributes associated with the aqueous polymeric composition 104 may be stored in the data base 208 associated with the user of the aqueous polymeric composition 104 as signified by arrow 220.

Through the output identifier or decentral identifier, the environmental attributes can be uniquely associated with the aqueous polymeric composition. Through the decentral network the environmental attributes may be transferred between the producer of the aqueous polymeric composition and the user of the aqueous polymeric composition. This way the environmental attributes can be shared with unique association to the aqueous polymeric composition and without central intermediary directly between the value chain players. This allows for transparency of environmental attributes across the value chain and positive environmental impacts from Aqueous polymeric composition of the chemical production network can be tracked through the value chain.

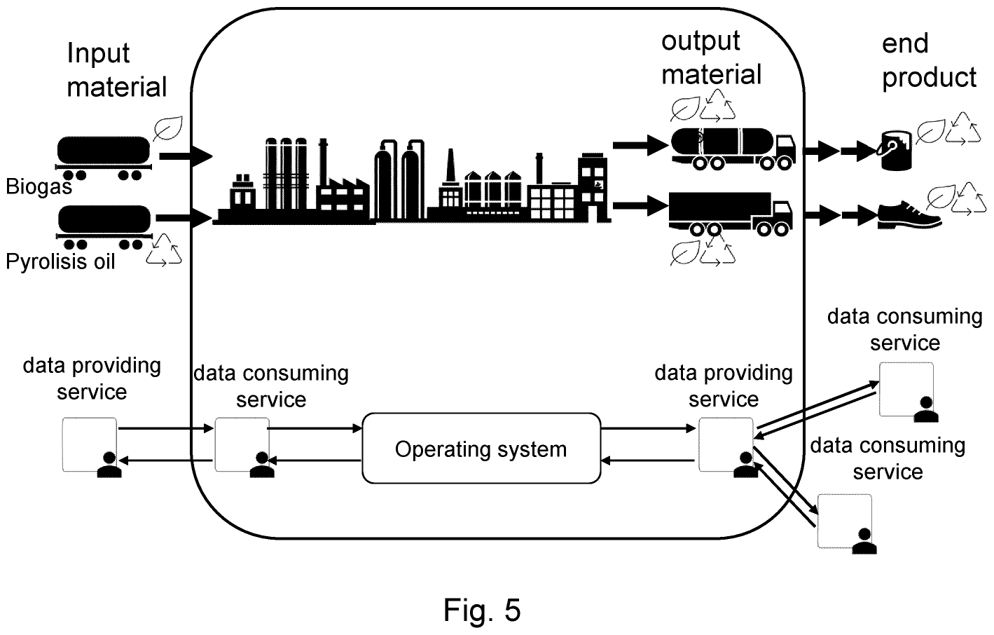

FIG. 5 illustrates schematically an example of a method or apparatus for providing environmental attributes associated with Aqueous polymeric composition across value chains via the decentral network.

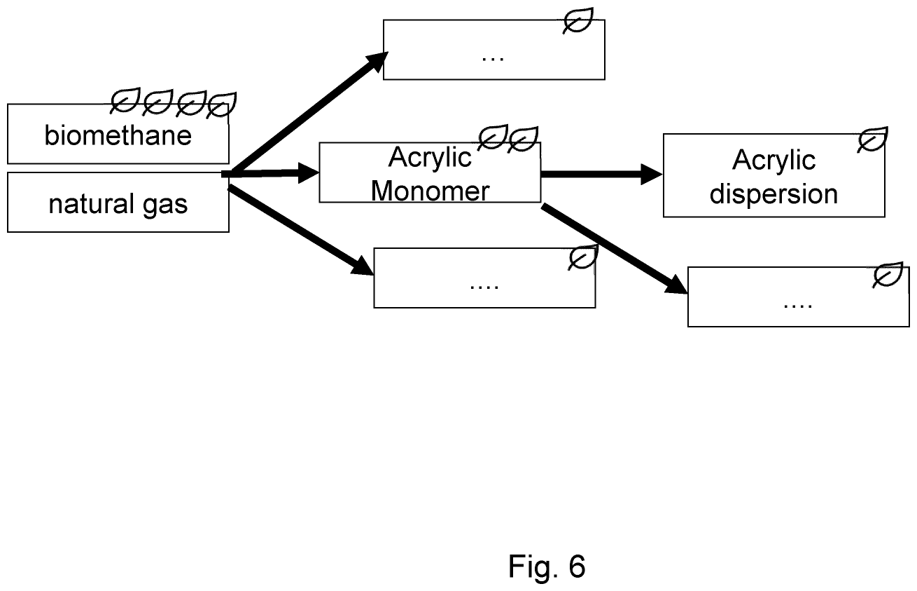

In the example of FIG. 6 a fully connected value chain including the chemical production network is illustrated. In the example, the input material provider, the aqueous polymeric composition producer, the aqueous polymeric composition user and the end product producer may be connected to the decentral network as described in the context of FIG. 4. Environmental attributes may be provided via the ID based schema described in the context of FIGS. 2-5 in the form of digital assets or Aqueous polymeric composition passports associated with the physical entity of the input material, the aqueous polymeric composition, any intermediate product or the end product.

The input material provider may provide the input materials such as bio-gas or pyrolysis oil. The environmental attributes of the input material may be provided through the data providing service connected to the decentral network as described in the context of FIG. 4. The Aqueous polymeric composition producer may produce the aqueous polymeric composition from the input material(s) provided to the chemical production network. The Aqueous polymeric composition producer may access the environmental attributes associated with the input material through a data consuming service connected to the decentral network as described in the context of FIG. 4. The Aqueous polymeric composition producer may manage the environmental attributes via the production operating system as described in the context of FIGS. 1 to 3. The Aqueous polymeric composition producer may assign the environmental attributes associated with the input materials or environmental attributes associated with the chemical production network such as the carbon footprint, to the aqueous polymeric composition as described in the context of FIGS. 1 to 3. The Aqueous polymeric composition producer may provide the environmental attributes associated with the aqueous polymeric composition through the data providing service connected to the decentral network as described in the context of FIG. 4. The Aqueous polymeric composition user or the end product producer may access the environmental attributes associated with the aqueous polymeric composition through the data consuming service connected to the decentral network as described in the context of FIG. 4.

The respective data owners in this example may be the input material producer, the output material producer, the output material user, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service may comprise computer-executable instructions for accessing and/or processing data, such as Aqueous polymeric composition data, associated with the data owner. The data providing service may comprise computer-executable instructions for providing and/or processing data, such as Aqueous polymeric composition data, associated with the data owner for accessing and/or processing by the data consuming service.

In the example of FIG. 4 the decentral identifier may relate to the end product. Such decentral identifier may be provided to the value chain participants. Via the end product specific decentral identifier data associated with the end product produced from the aqueous polymeric composition may be gathered across the production chain and assigned to the end product specific decentral identifier. For example, the one or more environmental attribute(s) associated with the end product may be derived from the environmental attribute(s) associated with the aqueous polymeric composition, the input material or any other product entity present in the value chain of the end product.

This way the environmental attributes of input materials, aqueous polymeric composition and any products produced from Aqueous polymeric composition may be tracked through the value chain up to the end product. By tracking the environmental attributes of materials in such way the information can be made transparent across the value chain while the information flow can be controlled by the participants in the supply chain. In addition, the environmental attributes can be handled according to the individual participants needs by production operating systems as described in the context of FIGS. 1 to 3. Overall, such tracking enables tracking of positive environmental impact by individual supply chain participants, which makes positive environmental impacts transparent and attributable to individual supply chain participants.

FIG. 6 illustrates schematically an example of a chemical production network for producing an aqueous polymer dispersion based product associated with the composition passport, or a passport providing regulatory information or a digital asset. The example of FIG. 6 is an illustrative example and should not be considered limiting.

In the example of FIG. 6 fossil feedstock, bio feedstock and recycled feedstock may be provided to the chemical production network for producing as an example a dispersion made from acrylic monomers (such as but not limited to (c1-c10) alkyl acrylate esters, acrylonitrile, acrylamide and/or acrylic acid, (called “acrylic dispersion”)), said monomers may be the input materials in this example as mixed feedstock. The acrylic dispersion may be used as component in a formulation containing at least one further component such as the other formulation compounds described before. By variation of these other formulation compounds different formulations may be produced. For example, using pigments, formulation additives and (inorganic) fillers as other formulation compounds, paints may be produced. Other formulations containing such aqueous polymer dispersions may lead to adhesives, paper coating chemicals, printing inks, overprint varnishes, laquers or sealing materials

In the example of FIG. 6 the chemical description and concentration of impurities in acrylic monomers as input material may be tracked. As described in the context of FIG. 2, the chemical description and concentration of impurities in acrylic monomers as input material at the start of the “acrylic dispersion” production chain may be registered via the production operating system 106. Here the chemical description and concentration of impurities in acrylic monomers as input material may be allocated to the balancing account 112 for chemical description and concentration of impurities in acrylic monomers as input material. The environmental attribute of chemical description and concentration of impurities in acrylic monomers as input material is hence detached from the mass flows of the chemical processing in the chemical production network 102 as illustrated in FIG. 6.

For attribution of the chemical description and concentration of impurities in acrylic monomers as input material used to produce the “acrylic dispersion”, the chemical description and concentration of impurities in acrylic monomers as input material may be determined. The chemical description and concentration of impurities in acrylic monomers as input material attributable to “acrylic dispersion” production may be based on mass conservation attributable to the produced “acrylic dispersion”. For example, only half of the chemical description and concentration of impurities in acrylic monomers as input material may be attributable to the “acrylic dispersion” and the other half may be attributable to other output products resulting from the acrylic monomer as input material. The environmental attribute chemical description and concentration of impurities in acrylic monomers as input material may be attributed to such extend to the “acrylic dispersion”.

In the system shown in FIG. 2, the environmental attribute associated with the production of “acrylic dispersion” may be attributed to the produced “acrylic dispersion” by associating the decentral ID to the “acrylic dispersion” and assigning the environmental attribute to the decentral ID. By linking the ID and the environmental attribute, the environmental attribute may be uniquely linked to the produced “acrylic dispersion”. The “acrylic dispersion” may be delivered to the “acrylic dispersion” user. The packaging such as lose bags with “acrylic dispersion” may include a QR-code. The decentral ID may be included or encoded to the QR code. This way the “acrylic dispersion” user may access the environmental attributes associated to the “acrylic dispersion” via the ID based protocol described in the context of FIGS. 4 and 5.

Similarly to this example the chemical production network for producing “acrylic dispersion”s associated with the digital asset may be based on the method illustrated in FIG. 3 for carbon footprints.

1. A system for producing an aqueous polymeric composition associated with a digital asset, the system comprising:
a chemical production network configured to produce the aqueous polymeric composition wherein the aqueous polymeric composition is produced from one or more input material(s) through one or more chemical process(s) of the chemical production network, wherein the one or more input material(s) and/or the one or more chemical process(s) are associated with environmental attribute(s);
a production operating apparatus configured to generate the digital asset by
providing a decentral identifier associated with the produced aqueous polymeric composition and one or more environmental attribute(s) of the one or more input material(s) and/or the one or more chemical process(s);
linking the decentral identifier and the environmental attribute(s); and
a chemical production network or system configured to provide each produced aqueous polymeric composition in association with the digital asset.

2. The system of claim 1, wherein the aqueous polymeric composition associated with the digital asset is a composition comprising emulsion polymers which comprise, in copolymerized form,
from 0.1 to 5% by wt. of at least one α,β-monoethylenically unsaturated mono- and/or dicarboxylic acid having from 3 to 6 carbon atoms and/or amide thereof and from 50 to 99.9% by wt. of at least one ester of acrylic and/or methacrylic acid with alkanols having from 1 to 20 carbon atoms and/or styrene, and optionally at least one further monomer
or
from 0.1 to 5% by wt. of at least one α,β-monoethylenically unsaturated
mono- and/or dicarboxylic acid having from 3 to 6 carbon atoms and/or amide thereof and
from 50 to 99.9% by wt. of styrene and/or butadiene,
and optionally at least one further monomer
or
from 0.1 to 5% by wt. of at least one α,β-monoethylenically unsaturated mono- and/or dicarboxylic acid having from 3 to 6 carbon atoms and/or amide thereof and from 50 to 99.9% by wt. of vinyl chloride and/or vinylidene chloride, and optionally at least one further monomer
or
from 0.1 to 5% by wt. of at least one α,β-monoethylenically unsaturated mono- and/or dicarboxylic acid having from 3 to 6 carbon atoms and/or amide thereof and from 50 to 99.9% by wt. of vinyl acetate, vinyl propionate, vinyl esters of versatic acid, vinyl esters of long-chain fatty acids, and/or ethylene, and optionally at least one further monomer.

3. The system of claim 1, wherein the digital asset of the aqueous polymeric composition includes information of impurities related to the input material(s).

4. The system of claim 1, wherein the one or more environmental attribute(s) associated with the aqueous polymeric compositions are provided from at least one balancing account configured to store environmental attribute(s) associated with input material(s).

5. The system of claim 1, wherein the one or more environmental attribute(s) are associated with at least one property related to the environmental impact of the one or more input material(s) and/or the chemical process(s).

6. The system of claim 1, wherein the production operating apparatus is configured to gather environmental attributes associated with the produced aqueous polymeric composition before, during and/or after production of the aqueous polymeric composition by the chemical production network.

7. The system of claim 1, wherein the environmental attribute(s) associated with the aqueous polymeric composition produced through chemical processes from one or more input material(s) provided to the chemical production network include the environmental attribute(s) associated with the input material(s), the chemical process(es) and/or the chemical production network(s).

8. The system of claim 1, wherein the environmental attribute(s) associated with input material(s) are provided before, during and/or after production of the aqueous polymeric composition by the chemical production network, wherein the environmental attribute(s) associated with input material(s) are allocated to at least one balancing account before, during and/or after production of the aqueous polymeric composition by the chemical production network.

9. The system of claim 1, wherein the environmental attribute(s) associated with the produced aqueous polymeric composition relate to environmental properties generated from process data associated with the chemical processing of the input material(s) and/or energy data associated with the energy consumption of the chemical processing, wherein the environmental attribute(s) associated with the produced aqueous polymeric compositions are generated before, during and/or after production of the aqueous polymeric composition by the chemical production network.

10. A method for producing an aqueous polymeric composition associated with a digital asset, wherein the method comprises:
producing the aqueous polymeric composition from one or more input material(s) through one or more chemical process(s) of a chemical production network, wherein the one or more input material(s) and/or the one or more chemical process(s) are associated with environmental attribute(s);
generating the digital asset by providing a decentral identifier associated with the produced aqueous polymeric composition and one or more environmental attribute(s) associated with the one or more input material(s) and/or the one or more chemical process(s);
linking the decentral identifier and the one or more environmental attribute(s); and
providing the produced aqueous polymeric composition in association with the digital asset.

11. An aqueous polymeric composition associated with a digital asset including a decentral identifier associated with the aqueous polymeric composition and linked to one or more environmental attribute(s) of the one or more input material(s) and/or the one or more chemical process(s) used to produce the aqueous polymeric composition.

12. The aqueous polymeric composition of according to claim 11, wherein the aqueous polymeric composition is a composition comprising emulsion polymers which comprise, in copolymerized form,
from 0.1 to 5% by wt. of at least one α,β-monoethylenically unsaturated mono- and/or dicarboxylic acid having from 3 to 6 carbon atoms and/or amide thereof and from 50 to 99.9% by wt. of at least one ester of acrylic and/or methacrylic acid with alkanols having from 1 to 20 carbon atoms and/or styrene, and optionally at least one further monomer
or
from 0.1 to 5% by wt. of at least one α,β-monoethylenically unsaturated mono- and/or dicarboxylic acid having from 3 to 6 carbon atoms and/or amide thereof and from 50 to 99.9% by wt. of styrene and/or butadiene, and optionally at least one further monomer
or
from 0.1 to 5% by wt. of at least one α,β-monoethylenically unsaturated mono- and/or dicarboxylic acid having from 3 to 6 carbon atoms and/or amide thereof and from 50 to 99.9% by wt. of vinyl chloride and/or vinylidene chloride, and optionally at least one further monomer
or
from 0.1 to 5% by wt. of at least one α,β-monoethylenically unsaturated mono- and/or dicarboxylic acid having from 3 to 6 carbon atoms and/or amide thereof and 50 to 99.9% by wt. of vinyl acetate, vinyl propionate, vinyl esters of versatic acid, vinyl esters of long-chain fatty acids, and/or ethylene, and optionally at least one further monomer.

13. A method for generating a digital asset associated with an aqueous polymeric composition, wherein the aqueous polymeric composition is produced from one or more input material(s) through one or more chemical process(s) of a chemical production network, wherein the one or more input material(s) and/or the one or more chemical process(s) are associated with environmental attribute(s), the method comprising:
providing a decentral identifier associated with the produced aqueous polymeric composition and one or more environmental attribute(s) of the one or more input material(s) and/or the one or more chemical process(s) used to produce the aqueous polymeric composition;
linking the decentral identifier and the environmental attribute(s); and
providing the digital asset in association with the produced aqueous polymeric composition, wherein the environmental attribute(s) associated with the aqueous polymeric composition is made accessible to a user of the aqueous polymeric composition through the digital asset.

14. A digital asset as generated according to the method of claim 13.

15. A method for using the digital asset generated according to the method of claim 13, the method comprising using the digital asset in production of a product produced from the aqueous polymeric composition associated with the digital asset.

16. A method for using the aqueous polymeric composition of claim 11 associated with the digital asset, the method comprising using the digital asset for producing a product from the aqueous polymeric composition and deriving a digital asset associated with the product from the aqueous polymeric composition digital asset.
